(19) United States
(12) Patent Application Publication
(10) Pub. No.: US 2021/0323908 A1
(43) Pub. Date: Oct. 21, 2021

(54) PROCESS FOR PRODUCING TCA CYCLE INTERMEDIATE CONJUGATES

(71) Applicant: IMBRIA PHARMACEUTICALS, INC., Boston, MA (US)

(72) Inventor: Yuelie Lu, San Diego, CA (US)

(21) Appl. No.: 17/226,352

(22) Filed: Apr. 9, 2021

Related U.S. Application Data
(60) Provisional application No. 63/008,153, filed on Apr. 10, 2020.

(51) Int. Cl. C07C 227/18; A61K 9/00

(52) U.S. Cl. CPC C07C 227/18; C07B 2200/13; A61K 9/0053

(57) ABSTRACT

The invention provides a process for preparing a TCA cycle intermediate conjugated to an amino acid having the structure of a compound of Formula (X):

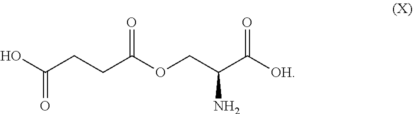 [SMILES: N[C@@H](COC(=O)CCC(=O)O)C(=O)O]

### CROSS-REFERENCE TO RELATED APPLICATIONS

This application claims the benefit of, and priority to, U.S. Provisional Application No. 63/008,153, filed Apr. 10, 2020, the contents of which are incorporated herein by reference.

### FIELD OF THE INVENTION

The invention relates to processes for producing tricarboxylic acid cycle (TCA) intermediates conjugated amino acids.

### BACKGROUND

Abnormal metabolism of the tricarboxylic acid (TCA) cycle (also known as the citric acid cycle or Krebs cycle) is associated with a variety of diseases, including inherited metabolic disorders, neurodegenerative diseases, and cancers. Inherited disorders of the TCA cycle cause intellectual disability, various neurological problems, and death in young children, while the neurodegenerative diseases and cancers that are coupled to dysfunction of the TCA cycle lead to cognitive and physical disabilities and death in adults.

Although the TCA cycle and its relationship to other intermediary metabolic pathways have been understood for decades, effective therapies for treating conditions associated with abnormal TCA cycle metabolism are lacking. Efforts to develop compositions that restore TCA cycle metabolism by delivering TCA cycle metabolites have been unsatisfactory. Compounds that provide unadulterated TCA cycle intermediates are challenging to administer orally due to the large amount of material that is needed to be taken by mouth and strong tastes or odors. Existing compositions are inadequate to remedy dysfunction of the TCA cycle, and people continue to suffer and die from a variety of conditions related to abnormal TCA cycle metabolism.

### SUMMARY

It was recently identified that conjugating amino acids to TCA cycle intermediates dramatically increases the solubility of those compounds. By incorporating such intermediates into higher solubility molecules, such molecules can then be metabolized to release the intermediates in the body. Provided herein is a process for producing TCA cycle intermediates conjugated to amino acids. An exemplary TCA cycle intermediate, optionally produced by the process of the present invention, has the structure of a compound of Formula (X):

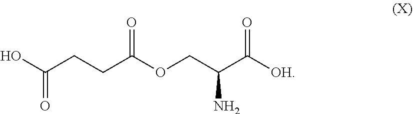 [SMILES: N[C@@H](COC(=O)CCC(=O)O)C(=O)O]

The compound of Formula (X) was recently identified as a promising therapeutic candidate for treating conditions associated with abnormal TCA cycle metabolism. A process to synthesize a compound of Formula (X) may comprise one or more of a protecting step, an esterification step, and a deprotecting step.

The process for producing the compound of Formula (X) may start with a compound that has the structure of the Formula (I):

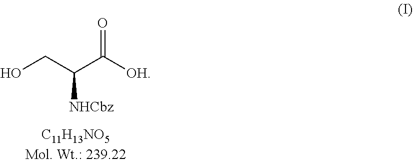 [SMILES: O=C(N[C@@H](CO)C(=O)O)OCc1ccccc1]

The process may comprise the step of protecting the compound of Formula (I). The protecting step may comprise preparing or providing a mixture comprising the compound of Formula (I), 1,8-Diazabicyclo[5.4.0]undec-7-ene (“DBU”) and Benzyl bromide (BnBr) to produce the compound of Formula (II):

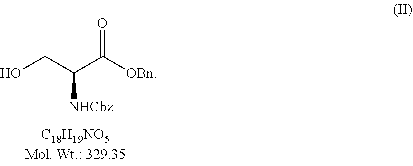 [SMILES: O=C(N[C@@H](CO)C(=O)OCc1ccccc1)OCc1ccccc1]

The process may comprise an esterification step. The esterification step may comprise preparing or providing a mixture comprising the compound of Formula (II) and a compound of Formula (III):

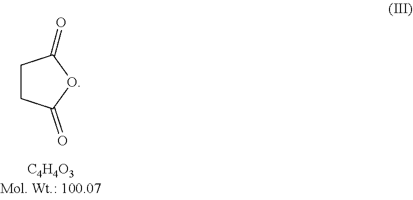

The esterification step may comprise preparing or providing a mixture comprising the compound of Formula (II) and the compound of Formula (III) to produce the compound of Formula (IV):

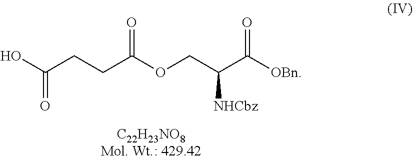 [SMILES: O=C(O)CCC(=O)OC[C@H](NC(=O)OCc1ccccc1)C(=O)OCc1ccccc1]

The esterification step may comprise preparing or providing a mixture comprising the compound of Formula (II) and the compound of Formula (III) in a mixture comprising pyridine and/or 4-Dimethylaminopyrodine (DMAP). The esterification step may comprise preparing or providing a mixture comprising the compound of Formula (II), the compound of Formula (III), pyridine, and DMAP in tetrahydrofuran (THF).

The process may comprise a deprotecting step. The deprotecting step may comprise preparing or providing a mixture comprising the compound of Formula (IV) and Pd(OH)₂/C in THE to form the compound of Formula (X):

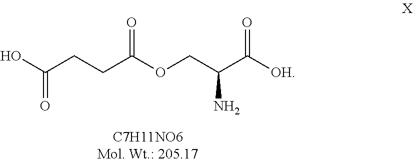 [SMILES: N[C@@H](COC(=O)CCC(=O)O)C(=O)O]

The deprotecting step may comprise preparing or providing a mixture comprising 15% Pd(OH)₂/C and the compound of Formula (IV) in THF. The deprotecting step may comprise stirring the mixture under H₂.

The deprotecting step may comprise forming the intermediate compound having the Formula (V):

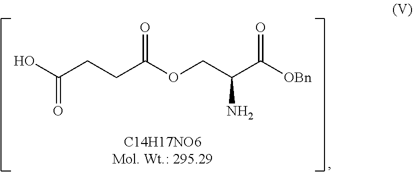

prior to forming the compound of Formula (X). The deprotecting step may produce methyl benzene and CO₂ in addition to the compound of Formula (X).

The compound of Formula (X) produced by the process of the invention is a crystalline polymorph identified as Type A.

The present invention provides for each of a compound of Formula (I), a compound of Formula (II), a compound of Formula (III), a compound of Formula (IV), a compound of Formula (V), and a compound of Formula (X). The present invention also provides for processes for preparing a compound of Formula (II), a compound of Formula (IV), a compound of Formula (V), and a compound of Formula (V).

The invention recognizes that the compound of Formula (X) is useful for the manufacture of pharmaceutical compositions. For example, pharmaceutical compositions that contain the Type A polymorph produced by the process of the present invention do not require special handling during storage or distribution.

Because the compound of Formula (X) produced by the process of the invention is water-soluble, it is useful as a therapeutic agent for treating conditions associated with abnormal TCA cycle metabolism. Due to the high solubility of the compounds, they are readily absorbed, circulate throughout the body, and can be cleaved to make the TCA cycle intermediate available to target tissues. In addition, the compound of Formula (X) is suitable for oral administration because the covalent linkages eliminate the taste or odor produced by free TCA cycle intermediates. Thus the compound of Formula (X) produced by the process of the invention also results in better patient compliance with a therapeutic regimen compared to formulations that use free TCA cycle intermediates.

The invention also provides methods of treating conditions associated with abnormal TCA cycle metabolism using compounds of Formula (X) produced by the process of the invention. In another aspect, the invention provides pharmaceutical compositions that include the compound of Formula (X) or a prodrug thereof.

The composition may be formulated for any route or mode of administration. The composition may be formulated for buccal, dermal, enteral, intraarterial, intramuscular, intraocular, intravenous, nasal, oral, parenteral, pulmonary, rectal, subcutaneous, topical, or transdermal administration. The composition may be formulated for administration by injection or with or on an implantable medical device (e.g., stent or drug-eluting stent or balloon equivalents). Preferably the composition is formulated for oral administration.

The composition may be formulated as a single unit dosage. The composition may be formulated as divided dosages.

The composition may contain a defined dose of the compound. The dose may contain from about 10 mg to about 2000 mg, from about 10 mg to about 1000 mg, from about 10 mg to about 800 mg, from about 10 mg to about 600 mg, from about 10 mg to about 400 mg, from about 10 mg to about 300 mg, from about 10 mg to about 200 mg, from about 25 mg to about 2000 mg, from about 25 mg to about 1000 mg, from about 25 mg to about 800 mg, from about 25 mg to about 600 mg, from about 25 mg to about 400 mg, from about 25 mg to about 300 mg, about 25 mg to about 200 mg, from about 50 mg to about 2000 mg, from about 50 mg to about 1000 mg, from about 50 mg to about 800 mg, from about 50 mg to about 600 mg, from about 50 mg to about 400 mg, from about 50 mg to about 300 mg, about 50 mg to about 200 mg, from about 100 mg to about 2000 mg, from about 100 mg to about 1000 mg, from about 100 mg to about 800 mg, from about 100 mg to about 600 mg, from about 100 mg to about 400 mg, from about 100 mg to about 300 mg, about 100 mg to about 200 mg, from about 200 mg to about 2000 mg, from about 200 mg to about 1000 mg, from about 200 mg to about 800 mg, from about 200 mg to about 600 mg, from about 200 mg to about 400 mg, from about 200 mg to about 300 mg, from about 300 mg to about 2000 mg, from about 300 mg to about 1000 mg, from about 300 mg to about 800 mg, from about 300 mg to about 600 mg, or from about 300 mg to about 400 mg of the compound. The dose may contain about 10 mg, about 25 mg, about 50 mg, about 100 mg, about 200 mg, about 300 mg, or about 400 mg of the compound.

In another aspect, the invention provides methods of treating a condition in a subject by providing to a subject having, or at risk of developing, a condition a composition containing a therapeutically effective amount of a compound of Formula (X). The composition may have any of the properties described above in relation to compositions that include the compound of Formula (X), including crystals of the compound.

The composition may be provided by any suitable route or mode of administration. The composition may be provided buccally, dermally, enterally, intraarterially, intramuscularly, intraocularly, intravenously, nasally, orally, parenterally, pulmonarily, rectally, subcutaneously, topically, transdermally, by injection, or with or on an implantable medical device (e.g., stent or drug-eluting stent or balloon equivalents). Preferably the composition is provided orally.

The composition may be provided as a single unit dosage. The composition may be provided as divided dosages. The composition may be provided in one dose per day. The composition may be provided in multiple doses per day. The composition may be provided in two, three, four, five, six, eight, or more doses per day.

The composition may contain a defined dose of the compound, such as any of the doses described above. The dose or doses may be provided for a defined period. One or more doses may be provided daily for at least one week, at least two weeks, at least three weeks, at least four weeks, at least six weeks, at least eight weeks, at least ten weeks, at least twelve weeks or more.

The condition may be a condition associated with abnormal TCA cycle metabolism. The condition associated with altered TCA cycle metabolism may be an inherited disorder, such as 2-oxoglutaric aciduria, fumarase deficiency, or succinyl-CoA synthetase efficiency. The condition associated with altered TCA cycle metabolism may be a neurodegenerative disorder, such as Amyotrophic Lateral Sclerosis, Alzheimer's disease, Parkinson's disease, or Huntington's disease. The condition associated with altered TCA cycle metabolism may be a cancer, such as pancreatic cancer, kidney cancer, cervical cancer, prostate cancer, muscle cancer, gastric cancer, colon cancer, glioblastoma, glioma, paraganglioma, leukemia, liver cancer, breast cancer, carcinoma, neuroblastoma.

The condition associated with altered TCA cycle metabolism may be an energetic disorder, refractory epilepsy, propionic academia (PA), methylmalonic academia (MMA), a long chain fatty acid oxidation disorder, succinyl CoA lyase deficiency, pyruvate carboxylase deficiency, mitochondrial respiratory chain deficiency, glutaric academia type 1 or type 2 a neurological disease, disorder or condition, a pain or fatigue disease, muscular dystrophy (e.g. Duchenne's muscular dystrophy, and Becker's muscular dystrophy), mitochondrial myopathy, mitochondrial encephalomyopathy lactic acidosis and stroke-like syndrome (MELAS), myoclonic epilepsy and ragged-red fibers (MERRF), a mitochondrial associated disease, or a disorder related to POLG mutation.

### BRIEF DESCRIPTION OF THE DRAWINGS

FIG. 1 is an HPLC readout following the reaction of a compound of Formula (I) to produce compound of Formula (II).

FIG. 2 is an HPLC readout following the reaction of a compound of Formula (I) to produce compound of Formula (II).

FIG. 3 is an HPLC readout following the reaction of a compound of Formula (I) to produce compound of Formula (II).

FIG. 4 is an HPLC readout following the reaction of a compound of Formula (I) to produce compound of Formula (II).

FIG. 5 is an HPLC readout following the reaction of a compound of Formula (I) to produce compound of Formula (II).

FIG. 6 is an HPLC readout following the reaction of a compound of Formula (II) and a compound of Formula (III) to produce a compound of Formula (IV).

FIG. 7 is an HPLC readout following the reaction of a compound of Formula (II) and a compound of Formula (III) to produce a compound of Formula (IV).

FIG. 8 is an HPLC readout following the reaction of a compound of Formula (II) and a compound of Formula (III) to produce a compound of Formula (IV).

FIG. 9 is an HPLC readout following the reaction of a compound of Formula (II) and a compound of Formula (III) to produce a compound of Formula (IV).

FIG. 10 is an HPLC readout following the reaction of a compound of Formula (IV) to produce a compound of Formula (X).

FIG. 11 is an HPLC readout following the reaction of a compound of Formula (IV) to produce a compound of Formula (X).

FIG. 12 is an HPLC readout following the reaction of a compound of Formula (IV) to produce a compound of Formula (X).

FIG. 13 is an HPLC readout following the reaction of a compound of Formula (IV) to produce a compound of Formula (X).

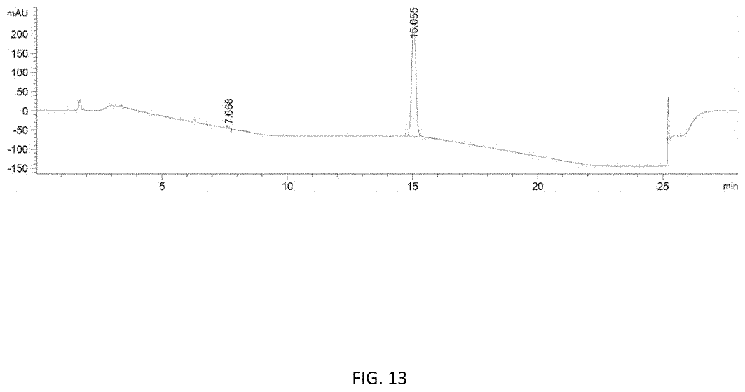

FIG. 14 is an HPLC readout following the reaction of a compound of Formula (IV) to produce a compound of Formula (X).

FIG. 15 is an HPLC readout following the reaction of a compound of Formula (IV) to produce a compound of Formula (X).

FIG. 16 is an HPLC readout following the reaction of a compound of Formula (IV) to produce a compound of Formula (X).

FIG. 17 is an HNM-R result of a reference standard for the compound of Formula (X).

FIG. 18 is an HNM-R result of a compound of Formula (X).

FIG. 19 is a chromatogram result an HPLC run comprising the compound of Formula (X).

FIG. 20 is a chromatogram result of a compound of Formula (X).

FIG. 21 shows a chromatogram result for the compound of Formula (X) and a standard.

FIG. 22 shows a chromatogram result for the compound of Formula (X) and serine.

FIG. 23 shows a chromatogram result for the compound of Formula (X) and succinic acid.

FIG. 24 shows a chromatogram result for two batches of the compound of Formula (X).

FIG. 25 shows a chromatogram result from a batch of the compound of Formula (X).

FIG. 26 shows a chromatogram result from a batch of the compound of Formula (X).

FIG. 27 shows a chromatogram result from a residual solvents assay of the compound of Formula (X).

### DETAILED DESCRIPTION

It has recently been identified that conjugating amino acids to TCA cycle intermediates or prodrugs dramatically increases the solubility of those compounds. Herein provided is a process for producing TCA cycle intermediates conjugated to amino acids. An exemplary TCA cycle intermediate, optionally produced by the process of the present invention, has the structure of a compound of Formula (X):

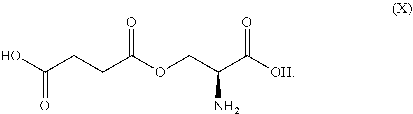 [SMILES: N[C@@H](COC(=O)CCC(=O)O)C(=O)O]

The compound of Formula (X) was recently identified as a promising therapeutic candidate for treating conditions associated with abnormal TCA cycle metabolism. A process to produce the compound of Formula (X) may comprise one or more of a protecting step, an esterification step, and a deprotecting step.

### Protecting Step

The process for preparing the compound of Formula (X) may start with a compound that has the structure of the Formula (I):

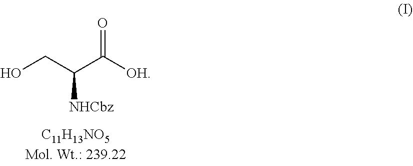 [SMILES: O=C(N[C@@H](CO)C(=O)O)OCc1ccccc1]

The process may comprise the step of protecting the compound of Formula (I). The protecting step may comprise preparing or providing a mixture comprising the compound of Formula (I), 1,8-Diazabicyclo[5.4.0]undec-7-ene (“DBU”) and Benzyl bromide (BnBr) to produce the compound of Formula (II):

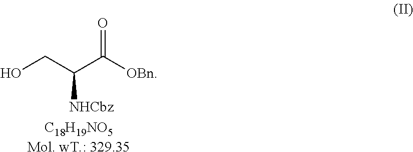

The protecting step may comprise preparing or providing a mixture of the compound of Formula (I), DBU, and BnBR in acetone. The protecting step may comprise stirring the mixture. The protecting step may comprise stirring the mixture at about 15° C. to about 25° C. The protecting step may comprise stirring the mixture at 15, 16, 17, 18, 19, 20, 21, 22, 23, 24, and/or 25° C. The protecting step may comprise stirring the mixture for at least 16 hours. After reaction completion the process may comprise adjusting the temperature of the mixture to about 40° C. to about 45° C. After reaction completion the process may comprise adjusting the temperature of the mixture to 40, 41, 42, 43, and/or 45° C. The process may further comprise concentrating the mixture under a vacuum. The protecting step may produce the reaction product of Formula (A) in addition to the compound of Formula (II)

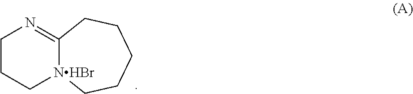

The protecting step may further comprise dissolving the mixture in ethyl acetate and water. The process may comprise stirring the resulting mixture. The process may comprise stirring the resulting mixture for at least about 30 minute. The process may further comprise separating an organic layer. The process may comprise washing the organic layer with NaHCO₃ and brine. The process may comprise concentrating the organic layer under a vacuum. The process may comprise providing to the mixture n-Heptane to form a slurry. The process may comprise filtering the slurry to form a wet cake and drying the wet cake under a vacuum. The wet cake may be dried at about 50° C. The compound of Formula (II) may be a white solid. The protection step may produce the compound of Formula (II) with at least a 94.5% yield, 99% chemical purity, and/or 99% ee chiral purity.

### Esterification Step

The present invention also provides a process of preparing a compound of Formula (X) from a compound of Formula (II). The process may comprise an esterification step. The esterification step may comprise preparing or providing a mixture comprising the compound of Formula (II) and a compound of Formula (III):

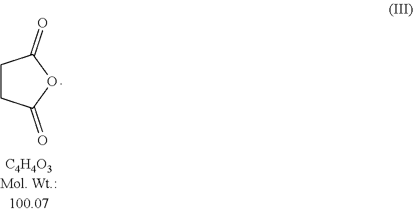

The esterification step may comprise preparing or providing a mixture comprising the compound of Formula (II) and the compound of Formula (III) to produce the compound of Formula (IV):

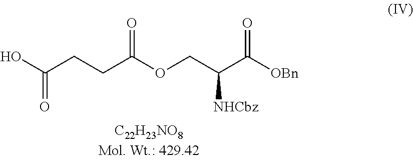 [SMILES: O=C(O)CCC(=O)OC[C@H](NC(=O)OCc1ccccc1)C(=O)OCc1ccccc1]

The esterification step may comprise preparing or providing a mixture comprising the compound of Formula (II) and the compound of Formula (III) in a mixture comprising pyridine and/or 4-Dimethylaminopyrodine (DMAP). The esterification step may comprise preparing or providing a mixture comprising the compound of Formula (II), the compound of Formula (III), pyridine, and DMAP in tetrahydrofuran (THF). The esterification step may comprise stirring the mixture. The esterification step may comprise stirring the mixture at about 15° C. to about 25° C. The esterification step may comprise stirring the mixture at 15, 16, 17, 18, 19, 20, 21, 22, 23, 24, and/or 25° C. The esterification step may comprise stirring the mixture for at least 20 hours. The process may further comprise adding to the mixture ethyl acetate and aqueous HCl. The process may comprise stirring the resulting mixture. The process may further comprise separating an organic layer. The process may comprise washing the organic layer with aqueous HCl and brine. The process may comprise concentrating the organic layer under a vacuum. The process may comprise concentrating the organic layer at a temperature of about 40° C. to about 45° C. The process may comprise concentrating the organic layer at a temperature of 40, 41, 42, 43, and/or 45° C. The process may comprise providing to the concentrated organic layer methyl tert-butyl ether (MTBE) to form a slurry. The process may comprise heating slurry to reflux. The process may comprise heating the slurry to reflux for at least 2 hours. The process may comprise cooling the heated slurry down to 20° C. filtered. The wet cake may be dried at about 50° C. The compound of Formula (IV) may be a white solid. The esterification may produce the compound of Formula (IV) with at least an 85% yield, 99% chemical purity, and/or 99% ee chiral purity.

### Deprotecting Step

The present invention also provides a process for preparing a compound of Formula (X) from a compound of Formula (IV). The process may comprise a deprotecting step. The deprotecting step may comprise preparing or providing a mixture comprising the compound of Formula (IV) and Pd(OH)₂/C in THE to form the compound of Formula (X):

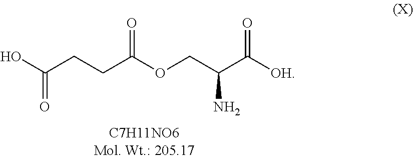 [SMILES: N[C@@H](COC(=O)CCC(=O)O)C(=O)O]

The deprotecting step may comprise preparing or providing a mixture comprising 15% Pd(OH)₂/C and the compound of Formula (IV) in THF. The deprotecting step may comprise stirring the mixture under H₂. The deprotecting step may comprise stirring the mixture under H₂ at about 0.3-0.4 MPa. The deprotecting step may comprise stirring the mixture at about 25° C. to about 30° C. The deprotecting step may comprise stirring the mixture at 25, 26, 27, 28, 29, and/or 30° C. The deprotecting step may further comprise filtering the mixture. The process may further comprise charging the filtered mixture with purified water to form a slurry. The process may further comprise heating the charged mixture to about 60° C. to about 70° C. The process may comprise stirring the mixture at 60, 61, 62, 63, 63, 64, 65, 66, 67, 68, 69, and/or 70° C. The process may comprise filtering the slurry through SiO₂, for example a pad of Celite® S, for example as sold by Sigma-Aldrich, St. Louis Mich. The process may comprise cooling the filtrated from about 5 to about 10° C. The process may comprise cooling the filtrate to 5, 6, 7, 8, 9, and/or 10° C. The filtrate may be stirred for at least 16 hours and may be further filtered. The filtered mixture may be washed with MTBE. The washed mixture may be dried at about 50° C. to about 60° C. The washed mixture may be dried at 50, 51, 52, 53, 54, 55, 56, 57, 58, 59, and/or 60° C. The deprotecting step may produce the compound of Formula (I) with at least a 75% yield, 95.6% chemical purity, and/or 99% ee chiral purity.

The deprotecting step may comprise forming the intermediate compound having the Formula (V):

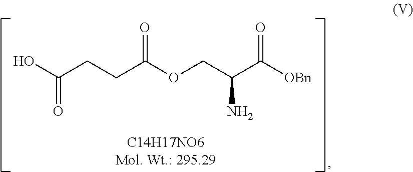

prior to forming the compound of Formula (X). The deprotecting step may produce methyl benzene and CO₂ in addition to the compound of Formula (X).

The present invention provides for each of a compound of Formula (I), a compound of Formula (II), a compound of Formula (III), a compound of Formula (IV), a compound of Formula (V), and a compound of Formula (X). The present invention also provides for processes for preparing a compound of Formula (II), a compound of Formula (IV), a compound of Formula (V), and a compound of Formula (V). A summary of the process of preparing each of a compound of Formula (II), a compound of Formula (IV), a compound of Formula (V), and a compound of Formula (X) starting with a compound of Formula (I) is shown below:

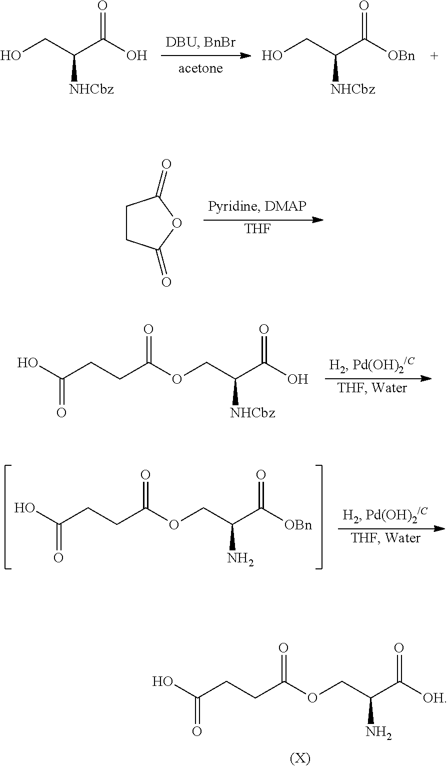

### Preparations of the Compound of Formula (X)

The compound of Formula (X), optionally prepared by the process of the invention, may be a crystalline polymorph identified as Type A. Crystals may be formed as salts of the compound of Formula (X). For example, crystals may be formed as hydrochloride salts of the compound of Formula (X) or as prodrugs of the compound of Formula (X).

Compounds of the invention may be provided as pharmaceutically acceptable salts, such as nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphrate, camphor sulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, striate, succinate, sulfate, tartrate, thiocyanate, p-toluoenesulfonate, undecanoate, valerate salts, and the like. Representative alkali or alkaline earth metal salts include sodium, lithium, potassium, calcium magnesium, and the like. In some embodiments, a pharmaceutically acceptable salt is an alkali salt. In some embodiments, a pharmaceutically acceptable salt is a sodium salt. In some embodiments, a pharmaceutically acceptable salt is an alkaline earth metal salt. In some embodiments, pharmaceutically acceptable salts include, when appropriate, nontoxic ammonium, quaternary ammonium, and amine cations formed using counter ions such as halide, hydroxide, carboxylate, sulfate, phosphate, nitrate, alkyl having from 1 to 6 carbon atoms, sulfonate, and aryl sulfonate.

### Pharmaceutical Compositions

The invention provides pharmaceutical compositions that contain the compound of Formula (X) or prodrugs thereof. The composition may include a hydrated form of the compound of Formula (X). The composition may include a monohydrate form of the compound of Formula (X). The composition may include an anhydrous form of the compound of Formula (X).

The composition may be formulated for any route or mode of administration. The composition may be formulated for buccal, dermal, enteral, intraarterial, intramuscular, intraocular, intravenous, nasal, oral, parenteral, pulmonary, rectal, subcutaneous, topical, or transdermal administration. The composition may be formulated for administration by injection or with or on an implantable medical device (e.g., stent or drug-eluting stent or balloon equivalents). Preferably the composition is formulated for oral administration.

The composition may be formulated as a single unit dosage. The composition may be formulated as divided dosages.

The composition may contain a defined dose of the compound of Formula (X). The dose may contain from about 10 mg to about 2000 mg, from about 10 mg to about 1000 mg, from about 10 mg to about 800 mg, from about 10 mg to about 600 mg, from about 10 mg to about 400 mg, from about 10 mg to about 300 mg, from about 10 mg to about 200 mg, from about 25 mg to about 2000 mg, from about 25 mg to about 1000 mg, from about 25 mg to about 800 mg, from about 25 mg to about 600 mg, from about 25 mg to about 400 mg, from about 25 mg to about 300 mg, about 25 mg to about 200 mg, from about 50 mg to about 2000 mg, from about 50 mg to about 1000 mg, from about 50 mg to about 800 mg, from about 50 mg to about 600 mg, from about 50 mg to about 400 mg, from about 50 mg to about 300 mg, about 50 mg to about 200 mg, from about 100 mg to about 2000 mg, from about 100 mg to about 1000 mg, from about 100 mg to about 800 mg, from about 100 mg to about 600 mg, from about 100 mg to about 400 mg, from about 100 mg to about 300 mg, about 100 mg to about 200 mg, from about 200 mg to about 2000 mg, from about 200 mg to about 1000 mg, from about 200 mg to about 800 mg, from about 200 mg to about 600 mg, from about 200 mg to about 400 mg, from about 200 mg to about 300 mg, from about 300 mg to about 2000 mg, from about 300 mg to about 1000 mg, from about 300 mg to about 800 mg, from about 300 mg to about 600 mg, or from about 300 mg to about 400 mg of the compound. The dose may contain about 10 mg, about 25 mg, about 50 mg, about 100 mg, about 200 mg, about 300 mg, or about 400 mg of the compound of Formula (X).

A pharmaceutical composition containing a compound of Formula (X) produced by the process of the invention may be in a form suitable for oral use, such as tablets, troches, lozenges, fast-melts, dispersible powders or granules, or capsules. Compositions intended for oral use may be prepared according to any method known in the art for the manufacture of pharmaceutical compositions and such compositions may contain one or more agents selected from sweetening agents, flavoring agents, coloring agents, and preserving agents, in order to provide pharmaceutically elegant and palatable preparations. Tablets contain the compound in admixture with non-toxic pharmaceutically acceptable excipients. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid, or talc. Preparation and administration of pharmaceutical compositions is discussed in U.S. Pat. No. 6,214,841 and U.S. Patent Publication No. 2003/0232877, the contents of each of which are incorporated by reference herein. Formulations for oral use may also be presented as hard gelatin capsules in which the compounds are mixed with an inert solid diluent, such as calcium carbonate, calcium phosphate or kaolin. The formulation may allow controlled release of the compound of Formula (X) in the gastrointestinal tract by encapsulating the compound in an enteric coating.

Dispersible powders and granules provide the compounds in admixture with a dispersing or wetting agent, suspending agent and one or more preservatives. Suitable dispersing or wetting agents and suspending agents are exemplified, for example, sweetening, flavoring, and coloring agents, may also be present.

The composition may comprise a prodrug of the compound of Formula (X). A prodrug is a medication or compound that, after administration, is metabolized (i.e. converted within the body) into a pharmaceutically active drug. The prodrug itself may be distributed, metabolized, and excreted. The prodrug may improve the bioavailability of the active drug when the active drug is poorly absorbed from the gastrointestinal tract. The prodrug may improve how selectively the drug interacts with cells or processes that are no its intended target, thereby reducing unintended and undesirable side effects. The prodrug may be converted into a biologically active form (bioactivated) inside cells (a Type I prodrug) or outside cells (a Type II prodrug). The prodrug may be bioactivated in the gastrointestinal tract, in systemic circulation, in metabolic tissue other than the target tissue, or in the target tissue. Thus, the compounds of the invention can be metabolized in the body to yield an intermediate of the TCA cycle.

### Providing a the Compound of Formula (X) to a Subject

The invention provides methods of treating a condition in a subject by providing the compound of Formula (X), optionally produced by the process of the invention. The compound of Formula (X) may be provided by any suitable route or mode of administration. For example and without limitation, the compound of Formula (X) produced by the process of the invention may be provided buccally, dermally, enterally, intraarterially, intramuscularly, intraocularly, intravenously, nasally, orally, parenterally, pulmonarily, rectally, subcutaneously, topically, transdermally, by injection, or with or on an implantable medical device (e.g., stent or drug-eluting stent or balloon equivalents).

The compound of Formula (X) may be provided according to a dosing regimen. A dosing regimen may include a dosage, a dosing frequency, or both.

Doses may be provided at any suitable interval. For example and without limitation, doses may be provided once per day, twice per day, three times per day, four times per day, five times per day, six times per day, eight times per day, once every 48 hours, once every 36 hours, once every 24 hours, once every 12 hours, once every 8 hours, once every 6 hours, once every 4 hours, once every 3 hours, once every two days, once every three days, once every four days, once every five days, once every week, twice per week, three times per week, four times per week, or five times per week.

The dose may contain a defined amount of the compound of Formula (X) that improves cardiac mitochondrial function, such as any of the doses described above in relation to pharmaceutical compositions containing the compound of Formula (X) produced by the process of the invention.

The dose may be provided in a single dosage, i.e., the dose may be provided as a single tablet, capsule, pill, etc. Alternatively, the dose may be provided in a divided dosage, i.e., the dose may be provided as multiple tablets, capsules, pills, etc.

The dosing may continue for a defined period. For example and without limitation, doses may be provided for at least one week, at least two weeks, at least three weeks, at least four weeks, at least six weeks, at least eight weeks, at least ten weeks, at least twelve weeks or more.

The subject may be a human. The subject may be a human that has a condition associated with abnormal TCA cycle metabolism. The subject may be a human that is at risk of developing a condition associated with abnormal TCA cycle metabolism. A subject may be at risk of developing a condition if the subject does not meet established criteria for diagnosis of the condition but has one or more symptoms, markers, or other factors that indicate the subject is likely to meet the diagnostic criteria for the condition in the future. The subject may be a pediatric, a newborn, a neonate, an infant, a child, an adolescent, a pre-teen, a teenager, an adult, or an elderly subject. The subject may be in critical care, intensive care, neonatal intensive care, pediatric intensive care, coronary care, cardiothoracic care, surgical intensive care, medical intensive care, long-term intensive care, an operating room, an ambulance, a field hospital, or an out-of-hospital field setting.

Conditions that May be Treated with a Compound of Formula (X)

The invention provides methods of treating a condition in a subject by providing a compound of Formula (X), optionally produced by the process of the invention. The condition may be any disease, disorder, or condition for which increasing mitochondrial energy production provides a therapeutic benefit.

The condition may be a condition associated with abnormal TCA cycle metabolism. For example and without limitation, the condition associated with altered TCA cycle metabolism may be an inherited disorder, such as 2-oxoglutaric aciduria, fumarase deficiency, or succinyl-CoA synthetase efficiency. The condition associated with altered TCA cycle metabolism may be a neurodegenerative disorder, such as Amyotrophic Lateral Sclerosis, Alzheimer's disease, Parkinson's disease, or Huntington's disease. The condition associated with altered TCA cycle metabolism may be a cancer, such as pancreatic cancer, kidney cancer, cervical cancer, prostate cancer, muscle cancer, gastric cancer, colon cancer, glioblastoma, glioma, paraganglioma, leukemia, liver cancer, breast cancer, carcinoma, neuroblastoma.

The condition associated with altered TCA cycle metabolism may be an energetic disorder, refractory epilepsy, propionic academia (PA), methylmalonic academia (MMA), a long chain fatty acid oxidation disorder, succinyl CoA lyase deficiency, pyruvate carboxylase deficiency, mitochondrial respiratory chain deficiency, glutaric academia type 1 or type 2 a neurological disease, disorder or condition, a pain or fatigue disease, muscular dystrophy (e.g. Duchenne's muscular dystrophy, and Becker's muscular dystrophy), mitochondrial myopathy, mitochondrial encephalomyopathy lactic acidosis and stroke-like syndrome (MELAS), myoclonic epilepsy and ragged-red fibers (MERRF), a mitochondrial associated disease, or a disorder related to POLG mutation.

### EXAMPLES

### Example 1

The compound of Formula (X) is prepared from a compound of Formula (I). First, a reactor containing a mixture of a compound of Formula (I), DBU, and BnBr in acetone is stirred at 15-25° C. for at least 16 hours to produce a compound of Formula (II). An IPC sample is pulled for reaction completion. Upon reaction completion (<90% (area %) of a compound of Formula (II) with respect to a compound of Formula (I)), the temperature of the mixture is adjusted to 40-45° C. The mixture is concentrated under vacuum until no liquid is dripped out. The residue is dissolved with ethyl acetate and water. The resulting solution is stirred at least 0.5 h and the organic layer is separated and washed with NaHCO₃ and brine. The organic layer is concentrated under vacuum and added by n-Heptane. The slurry is filtered, and the wet cake is dried under vacuum at 50° C. to produce a white solid. The reaction produced a 94.5% yield with 99% chemical purity and 99% ee chiral purity.

A reactor containing a mixture of the compound of Formula (II), a compound of Formula (III), pyridine, and DMAP in THE is stirred at 15-25° C. for at least 20 hours to produce a compound of Formula (IV). An IPC sample is pulled for reaction completion. Upon reaction completion (<90% (area %) of the compound of Formula (IV) with respect to the compound of Formula (II), ethyl acetate and aqueous HCl are added to the mixture and stirred. The organic layer is separated and washed with aqueous HCl and brine. The organic layer is concentrated under vacuum at 40-45° C., and MTBE is added to the residue. The resulting slurry is heated to reflux for at least 2 h and cooled down to 20° C. filtered. The wet cake is dried under vacuum at 50° C. to produce a white solid. The reaction produces an 85% yield with 99% chemical purity and 99% ee chiral purity.

A reactor containing a mixture of the compound of Formula (IV) and 15% Pd(OH)₂/C in THE is stirred under H₂ (0.3-0.4 MPa) at 25-30° C. for at least 20 hours to produce a compound of Formula (X) with a compound of Formula (V) as a possible intermediate. An IPC sample is pulled for reaction completion. Upon reaction completion (>99% (area %) consumption of the compound of Formula (IV) and the compound of Formula (V)), the mixture is filtered, and the wet cake is charged with purified water and heated to 60-70° C. The slurry is passed through a short pad of Celite® S, as sold by Sigma-Aldrich, St. Louis Mich., and is charged to the filtrate at 60-70° C. The mixture is passed through a short pad of Celite The filtrate is cooled to 5-10° C. and stirred for at least 16 h and filtered. The wet cake is washed with MTBE and dried at 50-60° to produce a white solid of a compound of Formula (X). The reaction produces a 73% yield with 95.6% chemical purity and 99% ee chiral purity.

Conclusions

The process for preparing a compound of Formula (X) from a compound of Formula (I) was highly efficient and reproducible. The polymorph of a compound of Formula (X) is a solid crystal polymorph identified as Type A that is highly stable and soluble.

### Example 2

Forming a Compound of Formula (II)

Two batches of the compound of Formula (X) were prepared from a compound of Formula (I). The first batch starting from 5.0 kg of compound of Formula (I) was given the identifier T0419-31 and the second batch starting from 40 kg of the compound of Formula (I) was given the identifier 201901.

For batch 201901, a 1500 L reactor was charged with 40 kg of a compound of Formula (I), 25.6 kg 1.0 eq of DBU, 30 kg 1.05 eq of BnBr and 250 kg of acetone and then stirred for about 16-20 hours at 15-25° C. for at least 16 hours to produce a compound of Formula (II). 52.6 kg of the compound of Formula (II) was produced.

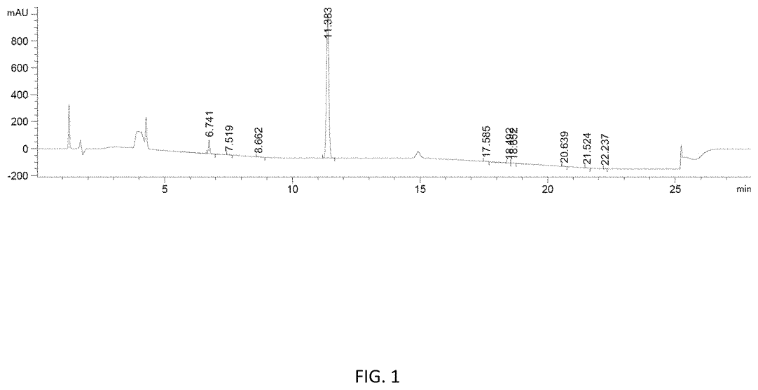

FIG. 1 shows the conversion percentage to a compound of Formula (II) as measured by HPLC of batch T0419-31.

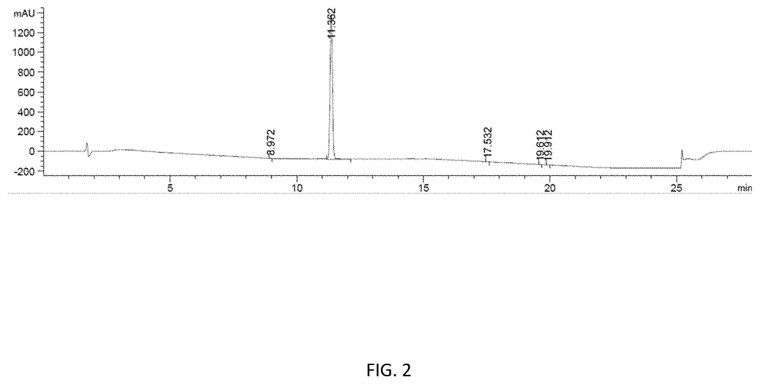

FIG. 2 shows the purity of the compound of Formula (II) as measured by HPLC of batch T0419-31.

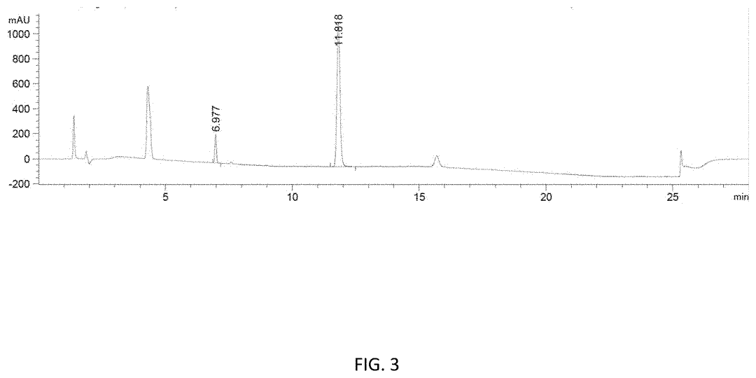

FIG. 3 shows the conversion percentage to a compound of Formula (II) as measured by HPLC of batch 201901 at this stage. The results indicate the conversion was 91.3% with a run time of 6.7 minutes and 8.7% for the compound of Formula (I) and a run time of 11.3 minutes and 91.3% for the compound of Formula (II).

The mixture was concentrated under vacuum at a temperature of 40-45° C. until no liquid dripped out. The residue was dissolved with 252 kg of ethyl acetate and 80 kg of water. The mixture was stirred for 0.5 hours. The organic layer was separated and washed with 85 kg of 5.8% aqueous NaHCO₃ and 96 kg of brine.

The organic layer was concentrated under vacuum at a temperature of 40-45° C. 220 kg of n-Heptane was added and stirred for 1 hour. The sully was filtered to afford a wet product which was dried under vacuum at 50° C. over 10 hours to give 52 kg of the compound of Formula (II), a white solid with a yield of 94.5% and a purity by HPLC of 99.9%.

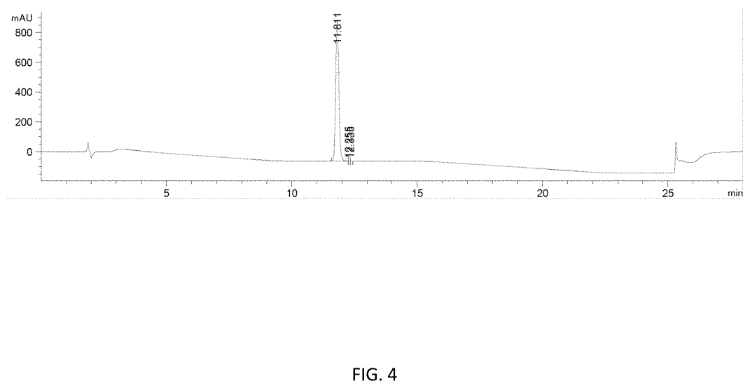

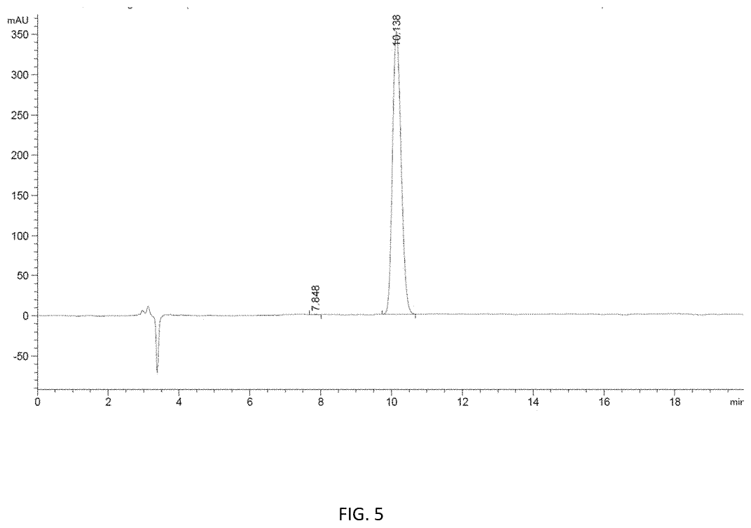

FIG. 4 and FIG. 5 show the purity of a compound of Formula (II) as measured by HPLC of batch 201901 at this stage A total of 52.4 kg of a compound of Formula (II) was made from 40 kg of a compound of Formula (I) with 99.9A % purity and a 94.5% yield. The yield of the manufacture is consistent to the non-GLP batch.

The potential impurity profile from the reaction is shown in Table 1.

The mass balance of a compound of Formula (II) produced is shown in Table 2.

TABLE 1
Potential impurity profile for the reaction producing Formula (II).

| | | | | | | |
|---|---|---|---|---|---|---|
| HPLC | 4.21 | 6.74 | 11.33 | 11.75 | 11.84 | 14.88 |
| RT (min) | | | | | | |
| RRT | 0.37 | 0.59 | 1.00 | 1.04 | 1.05 | 1.31 |
| | (DBU) | (SM1) | | | | (BrBn) |
| HPLC | N/A | N/A | 99.95% | 0.04% | 0.01% | N/A |
| (Area %) | | | | | | |
| Compound | | | | | | |
| Formula (I) | | | | | | |
| Structure | 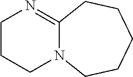 [SMILES: C1CCC2=NCCCN2CC1] | 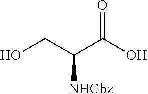 [SMILES: O=C(N[C@@H](CO)C(=O)O)OCc1ccccc1] | 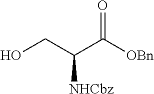 [SMILES: O=C(N[C@@H](CO)C(=O)OCc1ccccc1)OCc1ccccc1] | N/A | N/A | 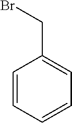 |

TABLE 2
Mass balance of a compound of Formula (II)

| | Input | Output | |
|---|---|---|---|
| | Compound of | Compound of | Mother liquid |
| Item | Formula (I) | Formula (II) | (n-heptane) |
| Mol | 167.4 | 159.3 | 5.0 |
| Ratio | 100% | 94.5% | 3.0% |

| | |
|---|---|
| Total recovery | 97.5% |

Results from the reaction are summarized in Table 3.

Reactants and reagents are summarized in Table 4.

HPLC retention times and details are summarized in Table 5.

TABLE 3
Summary of compound of Formula (II) formation by batch

| Starting | | | | | Quantity | | | | |
|---|---|---|---|---|---|---|---|---|---|
| compound | Conditions | | | | Compound | | | | |
| of Formula | | | Temperature/ | | of Formula | | | HPLC | |
| (I) | Reagent | Solvent | Time | Conversion % | (II) | | Yield | Purity | Batch ID |
| 5.0 kg | DBU | acetone | 15-25° C./ | 93.5% | 6.05 | kg | 87.9% | 99.9% | T0419-31 |
| (non-GLP) | (1.0 eq) | (8 V/W) | 16-20 h | | | | | | |
| 40 kg | BnBr | | | 91.3% | 52.4 | kg | 94.5% | 99.9%* | 201901 |
| (GLP) | (1.05 eq) | | | | | | | | |

*Loss on drying (LOD) from batch 201901 was 0.02% and Karl Fischer (KF) titration was 0.04%. Optical rotation for batch 201901 was +4.939 with a chiral purity of ee 99.9%.

TABLE 4
Summary of reactants and reagents

| | | | Quan- | | | Weight |
|---|---|---|---|---|---|---|
| | | | tity | | Molar | Ratio |
| Materials | Function | MW | (kg) | mol. | Ratio | (w/w) |
| Compound of | Reactant | 239.22 | 40 | 167.2 | 1.0 | 1.00 |
| Formula (I) | | | | | | |
| DBU | Reagent | 152.24 | 25.6 | 168.1 | 1.0 | 0.64 |
| BnBr | Reactant | 171.03 | 30 | 175.4 | 1.05 | 0.75 |
| Acetone | Reaction | 58.08 | 250 | — | — | 6.25 |
| | Solvent | | | | | |
| Ethyl acetate | Workup | 88.11 | 252 | — | — | 6.30 |
| | Solvent | | | | | |
| NaHCO₃ | Workup | 84.01 | 5 | — | — | 0.13 |
| | Reagent | | | | | |
| NaCl | Workup | 58.44 | 16 | — | — | 0.40 |
| | Reagent | | | | | |
| Water | Workup | 18.01 | 240 | — | — | 5.40 |
| | Reagent | | | | | |
| N-heptane | Workup | 100.2 | 220 | — | — | 5.50 |
| | Solvent | | | | | |

TABLE 5
HPLC retention times.

| HPLC RT (min) | 4.21 | 6.74 | 11.33 | 14.88 | 17.49 | 18.04 | 19.59 | 19.88 | 23.99 |
|---|---|---|---|---|---|---|---|---|---|
| RRT | 0.37 | 0.59 | 1.00 | 1.31 | 1.54 | 1.59 | 1.73 | 1.75 | 2.12 |
| | (DBU) | (SM1) | | (BnBr) | | | | | |
| Compound | DBU | Formula | Formula | BnBr | N/A | N/A | N/A | N/A | N/A |
| | | (I) | (II) | | | | | | |

Forming as Compound of Formula (IV)

The two batches of the compound of Formula (II) were then used to prepare a compound of Formula (IV). Batch T0419-31 started from 5.0 kg of the compound of Formula (II) and batch 201901 started from 52 kg of the compound of Formula (II).

For batch 201901, a 1500 L reactor was charged with 52 kg of a compound of Formula (II), 20.5 1.3 eq of a compound of Formula (III), 30.6 kg 2.5 eq of pyridine, 29 kg 1.5 eq of DMAP, and 360 kg of THF and then stirred for about 20-24 hours at 15-25° C. 57.8 kg of the compound of Formula (IV) was produced.

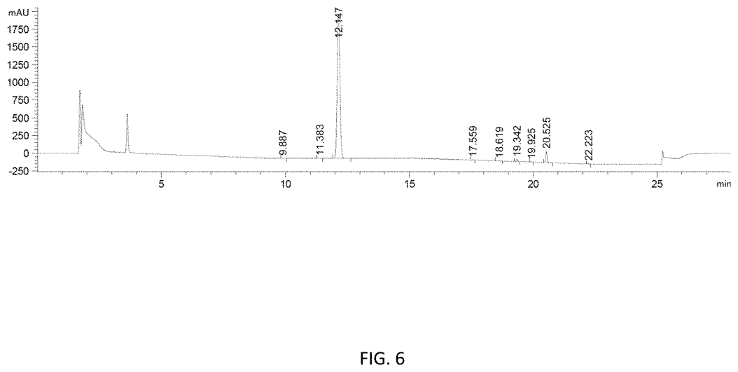

FIG. 6 shows the conversion percentage to a compound of Formula (IV) as measured by HPLC of batch T0419-31.

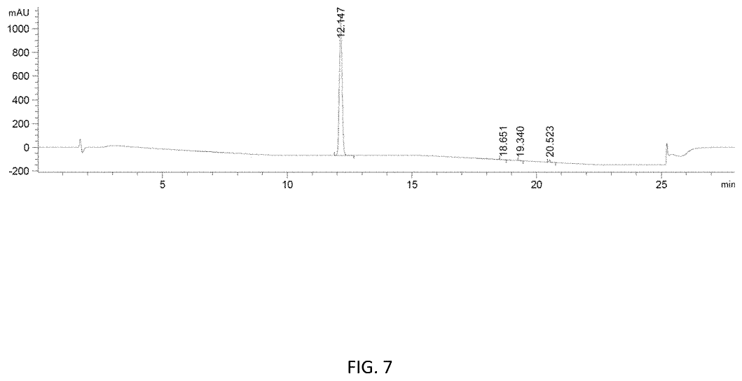

FIG. 7 shows the purity of the compound of Formula (IV) as measured by HPLC of batch T0419-31.

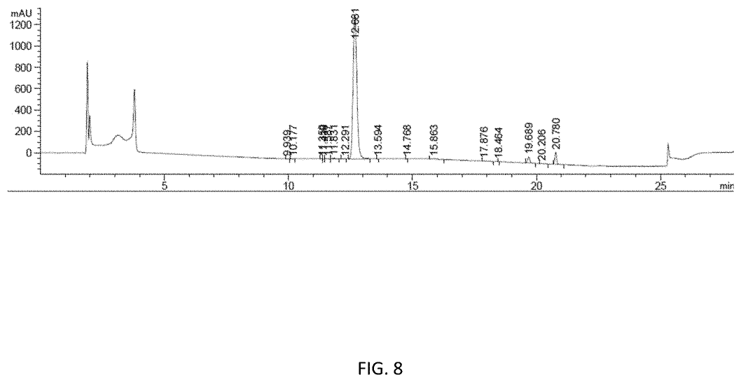

FIG. 8 shows the conversion percentage to a compound of Formula (IV) as measured by HPLC of batch 201901 at this stage. The results indicate the conversion was 99.9% with a run time of 11.1 minutes and 0.02% for the Compound of Formula (II) and a run time of 11.4 minutes and 93.3% for a compound of Formula (IV).

The reaction mixture was added with 280 kg of Ethyl acetate and 305 kg 1 mol/L aqueous HCl and stirred for 0.5 hours. The organic layer was separated and washed with 305 kg 1 mol/L of aqueous HCl and 340 kg of brine.

The organic layer was concentrated under vacuum at 40-45° C. 300 kg of MTBE was added to residue and heated to reflux for 2 hours, cooled at 20° C. and filtered to afford a wet product, which was dried at 50° C. over 10 hours to give 57.8 kg of a compound of Formula (IV), a white solid with a yield of 85.3% and a purity by HPLC of 99.1%.

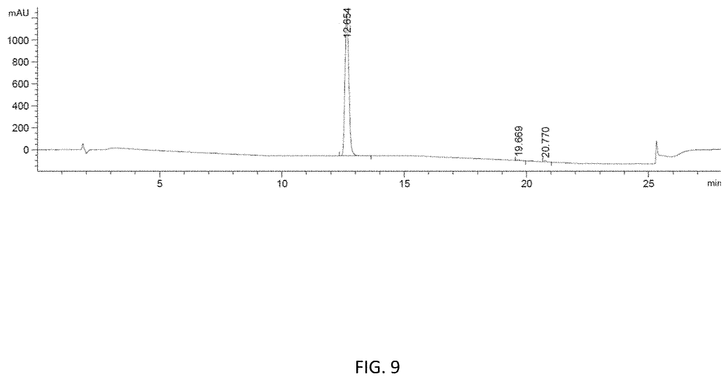

FIG. 9 shows the purity of a compound of Formula (II) as measured by HPLC of batch 201901 at this stage

A total of 57.8 kg of a compound of Formula (IV) was made from 52 kg of a compound of Formula (II), with 99.1% purity and a 85.3% yield. The yield of the manufacture is consistent to the non-GLP batch.

The potential impurity profile is shown in Table 6.

The mass balance of a compound of Formula (IV) produced is shown in Table 7.

TABLE 6
Potential impurity profile for the reaction producing Formula (IV).

| | | | | | | | |
|---|---|---|---|---|---|---|---|
| HPLC | 1.70 | 3.60 | 6.74 | 11.33 | 12.12 | 19.60 | 20.51 |
| RT | | | | | | | |
| (min) | | | | | | | |
| RRT | 0.14 | 0.30 | 0.56 | 0.93 | 1.00 | 1.62 | 1.69 |
| | | (DMAP) | Compound of | Compound of | | | |
| | | | Formula (I) | Formula (II) | | | |
| HPLC | N/A | N/A | N/A | N/A | 99.14 | 0.35 | 0.51 |
| Struc- ture | 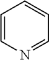 | 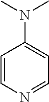 | 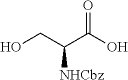 [SMILES: O=C(N[C@@H](CO)C(=O)O)OCc1ccccc1] | 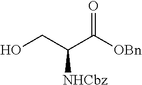 [SMILES: O=C(N[C@@H](CO)C(=O)OCc1ccccc1)OCc1ccccc1] | 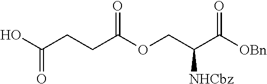 [SMILES: O=C(O)CCC(=O)OC[C@H](NC(=O)OCc1ccccc1)C(=O)OCc1ccccc1] | N/A | N/A |

TABLE 7
Mass balance of a compound of Formula (IV)

| | Input | Output | |
|---|---|---|---|
| | Compound of | Compound of | Mother liquid |
| Item | Formula (II) | Formula (IV) | (MTBE) |
| Mol | 158.1 | 134.7 | 15.8 |
| Ratio | 100% | 85.3% | 10% |

| | |
|---|---|
| Total recovery | 95.3% |

Results from the reaction are summarized in Table 8.

Reactants and reagents are summarized in Table 9.

HPLC retention times and details are summarized in Table 10.

TABLE 8
Summary of compound of Formula (IV) formation by batch

| Starting | | | | | Quantity | | | | |
|---|---|---|---|---|---|---|---|---|---|
| compound of | Conditions | | | | Compound | | | | |
| Formula | | | Temperature/ | | of Formula | | | HPLC | |
| (I) | Reagent | Solvent | Time | Conversion % | (IV) | | Yield | Purity | Batch ID |
| 5 kg | Compound of | THF | 15-25° C. | 99.8% | 5.6 | kg | 86.2% | 98.9% | T0419-41 |
| (non-GLP) | Formula (III) | (8 V/W) | 20-24 h | | | | | | |
| 52 kg | (1.3 eq) DMAP | | | 99.9% | 57.8 | kg | 85.3% | 99.1%* | 201901 |
| (GLP) | (1.5 eq) | | | | | | | | |
| | Pyridine | | | | | | | | |
| | (2.5 eq) | | | | | | | | |

*Loss on drying (LOD) from batch 201901 was 0.19% and Karl Fischer (KF) titration was 0.07%. Specific optical rotation for batch 201901 was +4.939 with a chiral purity of ee 99.9%.

TABLE 9
Summary of reactants and reagents

| | | | Quan- | | | Weight |
|---|---|---|---|---|---|---|
| | | | tity | | Molar | Ratio |
| Materials | Function | MW | (kg) | mol. | Ratio | (w/w) |
| Compound of | Reactant | 329.35 | 52 | 157.9 | 1.0 | 1.00 |
| Formula (I) | | | | | | |
| Compound of | Reactant | 100.07 | 20.5 | 204.9 | 1.3 | 0.39 |
| Formula (II) | | | | | | |
| DMAP | Reagent | 122.17 | 29 | 237.7 | 1.5 | 0.56 |
| Pyridine | Reagent | 79.1 | 30.6 | 386.9 | 2.5 | 0.59 |
| THF | Reaction | 72.11 | 360 | — | — | 6.92 |
| | solvent | | | | | |
| Ethyl acetate | Workup | 88.11 | 300 | — | — | 5.77 |
| | Solvent | | | | | |
| HCl | Workup | 36.46 | 50 | | | 0.96 |
| | Reagent | | | | | |
| NaCl | Workup | 58.44 | 60 | — | — | 1.15 |
| | Reagent | | | | | |
| Water | Workup | 18.01 | 840 | — | — | 16.15 |
| | Reagent | | | | | |
| MTBE | Workup | 88.15 | 300 | — | — | 5.77 |
| | Solvent | | | | | |

TABLE 10
HPLC retention times.

| HPLC RT (min) | 1.70 | 3.60 | 6.74 | 11.33 | 12.12 | 19.33 | 19.60 | 20.51 |
|---|---|---|---|---|---|---|---|---|
| RRT | 0.14 | 0.30 | 0.56 | 0.93 | 1.00 | 1.59 | 1.62 | 1.69 |
| | (pyridine) | (DMAP) | | | | | | |
| Compound | Pyridine | DMAP | Formula | Formula | Formula | N/A | N/A | N/A |
| | | | (I) | (II) | (IV) | | | |

Forming as Compound of Formula (X)

The two batches of the compound of Formula (IV) were then used to prepare four batches of a compound of Formula (X) given the batch IDs T0419-49, T0419-51, 201901 and 201902.

Batches T0419-49 and T0419-51 started from 2.2 kgs of the compound of Formula (IV). Batch 201901 started from 28.3 kg of a compound of Formula (IV). Batch 201902 started from 728.3 kg of a compound of Formula (IV).

For batch 201901, a 350 L reactor was charged with 28.3 kg of a compound of Formula (IV), 3.0 kg of 15% Pd(OH)₂, and 205 kg of THF. The mixture was degassed with H₂ for 3 times. The reaction mixture was stirred at 25-30° C. for 20 hours with about 0.3-0.4 Mpa H₂.

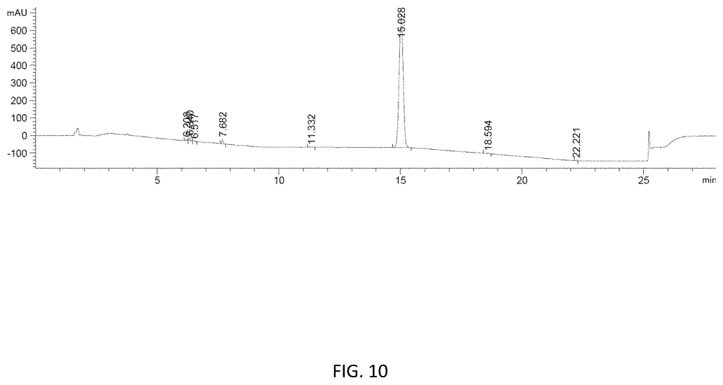

FIG. 10 shows the conversion percentage to an intermediate compound of Formula (V) as measured by HPLC of batch T0419-49.

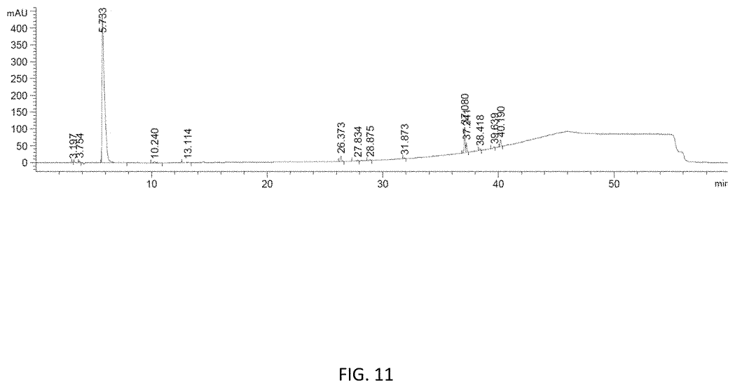

FIG. 11 shows the purity of the compound of Formula (X) as measured by HPLC of batch T0419-49.

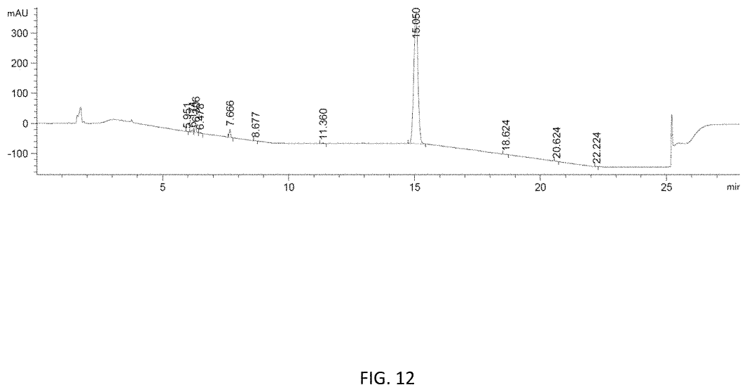

FIG. 12 shows the conversion percentage to an intermediate compound of Formula (V) as measured by HPLC of batch T0419-51.

FIG. 12 shows the conversion percentage to an intermediate compound of Formula (V) as measured by HPLC of batch T0419-51. The results indicate the conversion was 99.0% with a run time of 12.1 minutes for a compound of Formula (IC), and 7.6 minutes and 0.3% for the Compound of Formula (V).

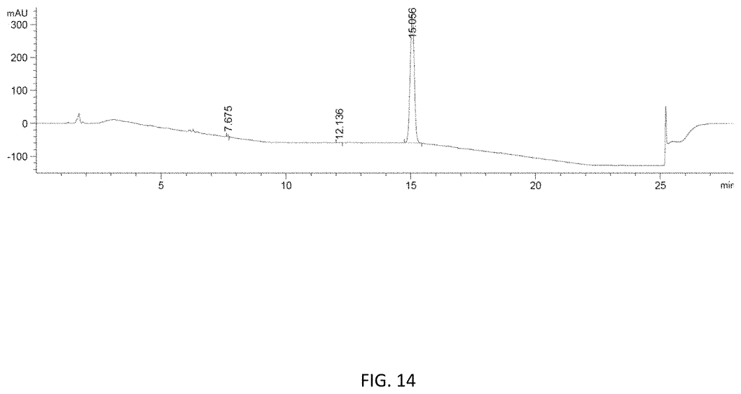

FIG. 14 shows the conversion percentage to a compound of Formula (X) as measured by HPLC of batch 201902 at this stage.

The reaction mixtures were filtered to remove solvents. The wet cake was charged to a reactor. 570 L of H₂O was added and heated to about 60-70° C. then filtered through a pad of fresh Ceilute. 6 kg of MS001 Silica Thiol was added to the filtrate at about 60-70° C., stirred for 1 hour, and filtered through a pad of fresh Celite.

The filtrate was cooled to about 5-10° C. and stirred for 16 hours, filtered, washed with 60 kg of MTBE, and dried at about 50-60° C. over 60 hours to give 20 kg of the compound of Formula (X), a white solid with a yield of 73.5% and Pd 10 ppm, a purity by HPLC of 97.3 and a chiral purity of 99.9% ee.

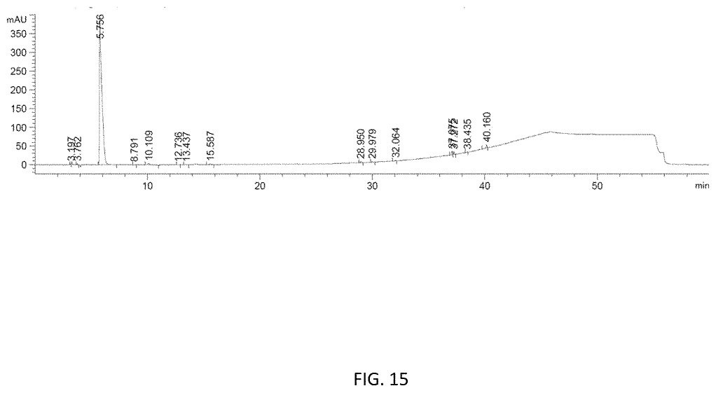

FIG. 15 shows the purity of a compound of Formula (X) as measured by HPLC of batch 201901 at this stage.

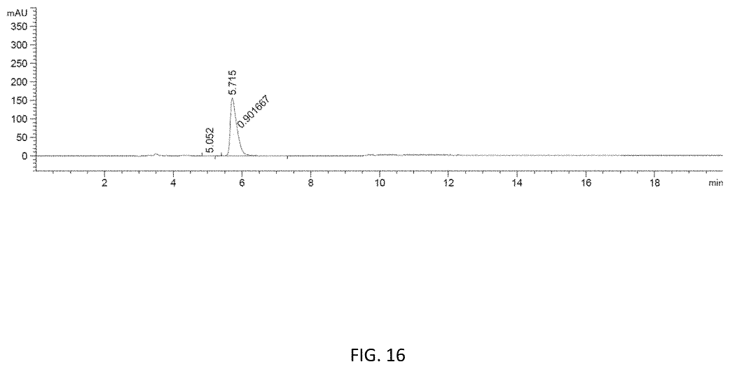

FIG. 16 shows the purity of a compound of Formula (X) as measured by HPLC of batch 201902 at this stage.

A total of 20 kg of a compound of Formula (X) was made from 56.6 kg of a compound of Formula (IV), with 98.0% and a 73.5% yield. The yield of the manufacture is consistent to the Lab batch. The product could be decomposed in water at 70-80° C.

The potential impurity profile is shown in Tables 11 and 12.

The mass balance of a compound of Formula (X) produced is shown in Table 13.

TABLE 11
Potential impurity profile for the reaction producing Formula (X).

| HPLC RT (min) | ~3.2 | ~4.2 | ~5.7 | ~14.3 | ~21.1 | ~26.1 | ~45.7 | ~45.7 |
|---|---|---|---|---|---|---|---|---|
| RRT | 0.59 | 0.74 | 1.00 | 2.43 | 3.69 | 4.38 | 7.94 | 7.98 |
| HPLC | 0.80 | N/A | 97.3 | 0.64 | N/A | N/A | N/A | N/A |
| (Area %) | | | | | | | | |
| Materials | Serine | Pyridine | Formula | Succinic | DMAP | DBU | BnBr | Toluene |
| | | | (X) | acid | | | | |

TABLE 12
Further potential impurity profile for
the reaction producing Formula (X).

| | | | | |
|---|---|---|---|---|
| HPLC | ~38.3 | ~40.1 | ~45.0 | ~45.3 |
| RT (min) | | | | |
| RRT | 6.69 | 6.98 | 7.86 | 7.92 |
| HPLC | 0.12 | N/A | N/A | N/A |
| (Area %) | | | | |
| Materials | Formula (I) | Formula (V) | Formula (II) | Formula (IV) |

TABLE 13
Mass balance of a compound of Formula (IV)

| | Input | Output | |
|---|---|---|---|
| | Compound of | Compound of | Mother liquid |
| Item | Formula (IV) | Formula (X) | (H₂O) |
| Mol | 131.9 | 97.6 | 29.0 |
| Ratio | 100% | 73.5% | 22.0% |

| | |
|---|---|
| Total recovery | 95.5% |

Results from the reaction are summarized in Table 14.

Reactants and reagents are summarized in Table 15.

HPLC retention times and details are summarized in Table 16.

TABLE 14
Summary of compound of Formula (X) formation by batch

| Starting | | | | | | | | | |
|---|---|---|---|---|---|---|---|---|---|
| compound | | | | | Quantity | | | | |
| of Formula | Conditions | | | | Formula | | | HPLC | |
| (IV) | Reagent | Solvent | Temp | Conversion %* | (X) | | Yield | Purity | Batch # |
| 2.2 kg | Pd(OH)₂/C | THF | 15-25° C. | Compound Formula | 1.5 | kg | 71.4% | 97.0% | T0419-49 |
| (Non-GLP) | (0.1 W/W | (8 V/W) | | (IV)/toluene: 0% | | | | Pd: 200 ppm | |
| | based on total) | | | Compound Formula | | | | | |
| | Pd(OH)₂ | | | (V)/toluene: 1.0% | | | | | |
| | assay: 14.5% | | | Compound Formula | | | | | |
| | | | | (X): 99.0% | | | | | |
| 2.2 kg | Pd(OH)₂/C | THF | 15-25° C. | Compound Formula | | | | | T0419-51 |
| (Non-GLP) | (0.1 W/W | (8 V/W) | | (IV)/toluene: 0% | | | | | |
| | based on total) | | | Compound Formula | | | | | |
| | Pd(OH)₂ | | | (V)/toluene: 2.0% | | | | | |
| | assay: 14.5% | | | Compound Formula | | | | | |
| | | | | (X): 97.9% | | | | | |
| 28.3 kg | Pd(OH)₂/C | THF | 25-30° C. | Formula | 20 | kg | 73.5% | 97.3% | 201901 |
| (GLP) | (0.1 W/W | (8 V/W) | | (IV)/toluene: 0% | | | | ee: 99.9% | |
| | based on total) | | | Formula | | | | Pd: 10 ppm | |
| | Pd(OH)₂ | | | (V)/toluene: 0.3% | | | | | |
| | assay: 14.5% | | | Formula (X): 99.6% | | | | | |
| 28.3 kg | Pd(OH)₂/C | THF | 25-30° C. | Formula | | | | | 201902 |
| (GLP) | (0.1 W/W | (8 V/W) | | (IV)/toluene: 0% | | | | | |
| | based on total) | | | Formula | | | | | |
| | Pd(OH)₂ | | | (V)/toluene: 0.47% | | | | | |
| | assay: 14.5% | | | Formula (X): 99.5% | | | | | |

*Run times: Compound of Formula (IV): 12.1 min; Compound of Formula (V): 7.6 min; toluene: 15.0 min; Compound of Formula (IV)/toluene ≤0.5%; Compound of Formula (V)/toluene ≤0.5%

TABLE 15
Summary of reactants and reagents

| | | | Quan- | | | Weight |
|---|---|---|---|---|---|---|
| | | | tity | | Molar | Ratio |
| Materials | Function | MW | (kg) | mol. | Ratio | (w/w) |
| Compound of | Reactant | 429.42 | 56.6 | 56.9 | 66.4 | 1.00 |
| Formula (IV) | | | | | | |
| 15% | Reagent | 140.43 | 6.0 (0.10 | — | — | 0.11 |
| Pd(OH)₂/C | | | w/w based on | | | |
| | | | total) Pd(OH)₂ | | | |
| | | | assay: 14.5% | | | |
| THF | Reaction | 72.11 | 410 | — | — | 7.24 |
| | solvent | | | | | |
| MS001 | Pd | — | 6 | — | — | 0.10 |
| | Scavenger | | | | | |
| Water | Workup | 18.01 | 570 | — | — | 10.07 |
| | solvent | | | | | |
| Celite | Workup | 60.08 | 120 | — | — | 2.12 |
| | Reagent | | | | | |
| MTBE | Workup | 88.15 | 60 | — | — | 1.06 |
| | Solvent | | | | | |

TABLE 16
HPLC retention times.

| HPLC RT (min) | ~3.2 | ~4.2 | ~5.7 | ~14.3 | ~21.1 | ~26.1 | ~38.3 | ~40.1 | ~45.0 | ~45.3 | ~45.7 | ~45.7 |
|---|---|---|---|---|---|---|---|---|---|---|---|---|
| RRT | 0.59 | 0.74 | 1.00 | 2.43 | 3.69 | 4.38 | 6.69 | 6.98 | 7.86 | 7.92 | 7.94 | 7.98 |
| Materials | Serine | Pyridine | Formula | Succinic | DMAP | DBU | Formula | Formula | Formula | Formula | BnBr | Toluene |
| | | | (X) | acid | | | (I) | (V) | (II) | (IV) | | |

### Example 3

### Quality Assays

The process for preparing a compound of Formula (X) from a compound of Formula (I) was highly efficient and reproducible. The polymorph of a compound of Formula (X) is a solid crystal polymorph identified as Type A that is highly stable and soluble. 2 g of the test sample was uniformly placed on white filter paper, visually under natural light for a visual inspection indicating an off-white solid. Results from batch 201902 were further analyzed by hydrogen nuclear magnetic resonance spectroscopy (H-NMR-D20) against a reference standard batch produced as T0419-34-03, and the produced batch conformed with the reference standard. Results from batch 201902 were further analyzed by liquid chromatography-mass spectroscopy (LCMS) against a reference standard batch produced as T0419-34-03, and the produced batch conformed with the reference standard.

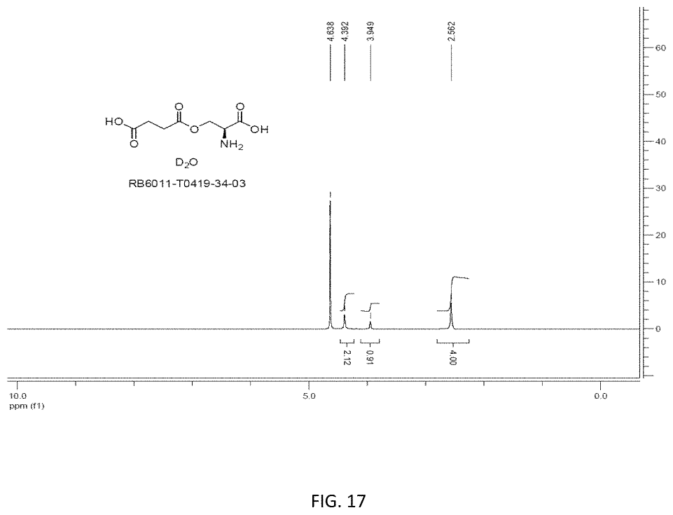

FIG. 17 is an HNM-R result for the reference standard T0419-34-03.

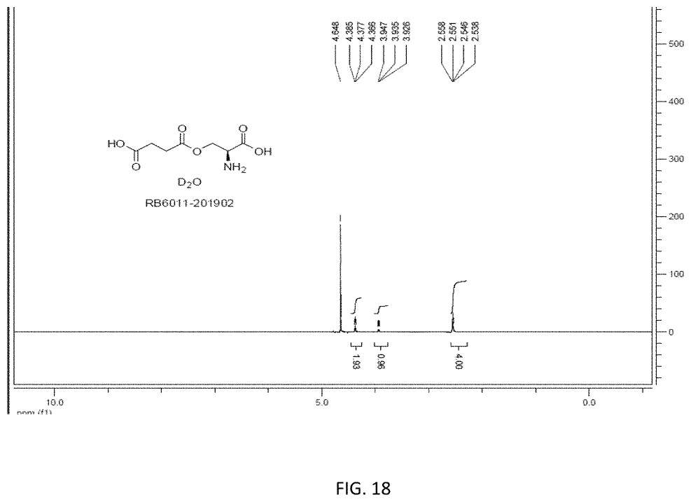

FIG. 18 is an HNM-R result for the compound of Formula (X) formed from batch 201902.

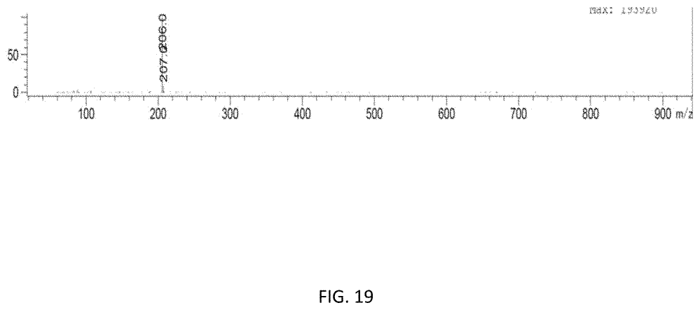

FIG. 19 is an LCMS result for the reference standard T0419-34-03.

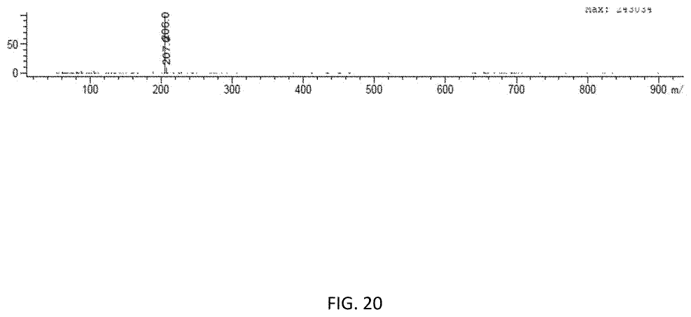

FIG. 20 is an LCMS result for the compound of Formula (X) formed from batch 201902.

The specific optical rotation in saturated sodium bicarbonate solution at 25° C. indicated a +25.570° result.

### HPLC Assay

Chemical purity was further analyzed by HPLC using distilled water, methanol, and phosphoric acid as solvents. A 150*4.6 mm 5 μm column sold under the trade name ECLIPSE XDB C18 by Agilent Technologies, Inc., headquartered in Santa Clara, Calif. was used. Methanol (solvent A) and 0.1% phosphoric acid in water (solvent B) was run according to the gradients shown in Table 17 at a flow rate of 0.5 mL/min, with a UV 210 nm detector, a column temperature of 35° C., an inject volume of 10 μL, and a run time of 60 minutes.

Two solutions were run using the diluent of 0.1% phosphoric acid and water. In solution 1, 100 mg of the compound of Formula (X) was placed in a 10 ml volumetric flask and diluted to volume with 0.1% Phosphoric acid in water. In solution 2, 100 mg of the compound of Formula (X) was placed into a 10 ml volumetric flask and diluted to volume with 0.1% Phosphoric acid in water.

TABLE 17
The gradient table for the HPLC run.

| Time (min) | A % | B % |
|---|---|---|
| 0 | 0 | 100 |
| 5 | 0 | 100 |
| 20 | 10 | 90 |
| 40 | 80 | 20 |
| 50 | 80 | 20 |
| 50.10 | 0 | 100 |
| 60 | 0 | 100 |

The following calculations were made based on the HPLC run:

### Calculation #1:

$$R C {F}_{Each impurity} = \frac{{C}_{Each impurity}}{{A}_{Each impurity}} \div \frac{{C}_{RB}}{{A}_{RB}}$$

### Where:

RCF_{Each impurity}: Relative correction factor of each impurity peak.

C_{Each impurity}: The concentration of impurity in each impurity reference solution;

C_{RB6011}: The concentration of the compound of Formula (X) in the reference solution;

A_{Each impurity}: Area of impurity in each impurity reference solution;

A_{RB6011}: Area of the compound of Formula (X) in the reference solution.

### Calculation #2:

Purity=(AsλRCF_{S})/(ΣA−_{Each impurity}×RCF_{Each impurity}+As×RCF_{S})×100%

### Where:

As: Area of RB6011

RCF_{S}: Relative correction factor of RB6011

A_{Each impurity}: Area of each Impurity

RCF_{Each impurity}: Relative correction factor of each impurity

Calculation details are summarized in Table 18.

TABLE 18
HPLC Calculation details for the compound of Formula (X)

| | | | | | Relative | |
|---|---|---|---|---|---|---|
| | | | | | correction factor | |
| | | | | | (RCF_{Each impurity} = | |
| Related | | C | | Response Factor | RF_{Each impurity}/ | |
| substances | RRT | mg/mL | Peak Area | (RF) = C/Peak Area | RF_{RB6011}) | Purity (%) |
| Serine | 0.59 | N/A | N/A | N/A | 0 | This impurity has been tested |
| | | | | | | and reported by LC-MS |
| | | | | | | method. |
| Pyridine | 0.74 | 0.0467 | 448.6151 | 1.041 × 10⁻⁴ | 3.701 × 10⁻² | N/A |
| RB6011 | 1.00 | 0.1744 | 61.99131 | 2.813 × 10⁻³ | 1 | N/A |
| Succinic acid | 2.43 | N/A | N/A | N/A | 0 | This impurity has been tested |
| | | | | | | and reported by LC-MS |
| | | | | | | method. |
| DMAP | 3.69 | 0.1526 | 5639.574 | 2.706 × 10⁻⁵ | 9.620 × 10⁻³ | N/A |
| DBU | 4.38 | 0.1549 | 2919.015 | 5.307 × 10⁻⁵ | 1.887 × 10⁻² | N/A |
| Impurity C | 4.61 | N/A | N/A | N/A | 8.614 × 10⁻² | Same as toluene |
| Impurity A | 6.44 | N/A | N/A | N/A | 2.778 × 10⁻² | Same as RB6011-IM295 |
| Impurity B | 6.48 | N/A | N/A | N/A | 2.778 × 10⁻² | Same as RB6011-IM295 |
| Formula (I) | 6.69 | 0.1879 | 33440.994 | 5.627 × 10⁻⁵ | 1.999 × 10⁻² | N/A |
| Formula (V) | 6.98 | 0.1662 | 2127.019 | 7.814 × 10⁻⁵ | 2.778 × 10⁻² | N/A |
| Formula (II) | 7.86 | 0.1904 | 4872.407 | 3.908 × 10⁻⁵ | 1.389 × 10⁻² | N/A |
| Formula (IV) | 7.92 | 0.1797 | 3512.98 | 5.115 × 10⁻⁵ | 1.818 × 10⁻² | N/A |
| BrBn | 7.94 | 0.4671 | 9629.706 | 4.851 × 10⁻⁵ | 1.724 × 10⁻² | N/A |
| Toluene | 7.98 | 0.0519 | 2141.632 | 2.423 × 10⁻⁵ | 8.614 × 10⁻³ | N/A |
| Other Impurity | N/A | N/A | N/A | N/A | 1 | N/A |

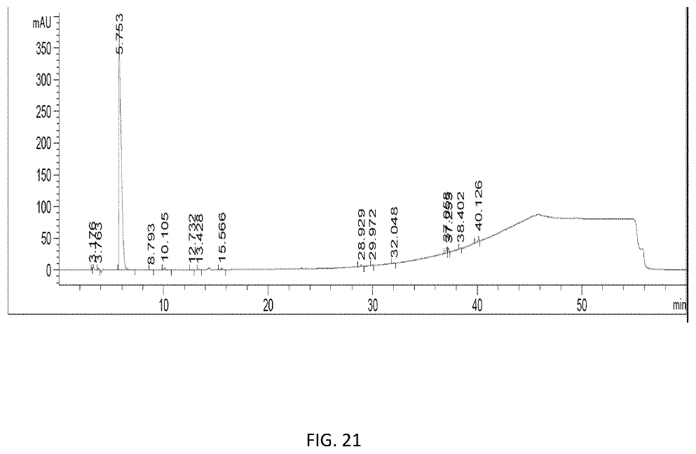

FIG. 21 shows a typical chromatogram result for the compound of Formula (X) generated from batch 201902.

HPLC purity results are summarized in Table 19.

TABLE 19
HPLC purity results for the compound of Formula (X)
Purity (HPLC, % a/a)

| Compound of Formula (X) | | 97.3% |
|---|---|---|
| Individual unknown | Impurity RRT 0.55 | 0.17% |
| impurity | Impurity RRT 0.65 | 0.20% |
| | Impurity RRT 1.53 | 0.12% |
| | Impurity RRT 1.76 | 0.65% |
| | Impurity RRT 2.21 | 0.13% |
| | Impurity RRT 2.33 | 0.15% |
| | Impurity RRT 2.71 | 0.44% |
| | Impurity RRT 5.03 | 0.37% |
| | Impurity RRT 5.21 | 0.16% |
| | Impurity RRT 5.57 | 0.23% |
| | Impurity RRT 6.68 | 0.12% |
| Individual known | Pyridine (RRT 0.74) | N.D |
| impurity | DMAP (RRT 3.69) | N.D |
| | DBU (RRT 4.38) | N.D |
| | Formula (I) (RRT 6.69) | N.D |
| | Formula (V) (RRT 6.98) | N.D |
| | Formula (II) (RRT 7.86) | N.D |
| | Formula (IV) (RRT 7.92) | N.D |
| | Toluene (RRT 7.98) | N.D |

### LC-MS Assay

Chemical purity was further analyzed by LC-MS using distilled water, acetonitrile, and formic acid as solvents. A 250*4.6 mm 5 μm column sold under the trade name ECLIPSE XDB C18 by Agilent Technologies, Inc., headquartered in Santa Clara, Calif. was used. Acetonitrile (solvent A) and 0.1% formic acid in water (solvent B) was run according to the gradients shown in Table 20 at a flow rate of 0.8 mL/min, with a column temperature of 25° C., an inject volume of 5 μL, and a run time of 15 minutes. A SIM model with a SIM Ion of 206, a positive polarity, and a fragmentor value of 70 was set.

TABLE 20
The gradient table for the LC-MS run.

| Time (min) | A % | B % |
|---|---|---|
| 0 | 95 | 5 |
| 8 | 5 | 95 |
| 11 | 5 | 95 |
| 11.1 | 95 | 5 |
| 15 | 95 | 5 |

With water as the diluent, a standard solution and sample solution were made. The standard solution comprises 20 mg of the standard batch T0419-34-03 in a 100 ml volumetric flask and dilute to volume with water. The sample solution comprises 20 mg of the compound of Formula (X) from batch 201902 in a 100 ml volumetric flask dilute to volume with water. The sequence of solution runs is shown in Table 21.

TABLE 21
Table of sequence runs for LC-MS analysis.

| | | Inject ion | |
|---|---|---|---|
| No. | Solution | number | System suitability criteria |
| 1 | Diluent | ≤1 | No interference |
| 2 | Working standard | 5 | RSD ≤ 5.0% |
| | solution 1 | | |
| 3 | Working standard | 1 | Recovery rate 95%-105% |
| | solution 2 | | |
| 4 | Test solution 1 | 1 | / |
| 5 | Test solution 2 | 1 | / |
| 6 | Working standard | 1 | Recovery rate 95%-105% |
| | solution 1 | | |

The following calculation was made based on the LC-MS run:

Assay (%)=(C_{STD1}×A_{SPL}×Pᵣ)/(C_{SPL}×A_{STD1})×100%

### Where

C_{STD1}: The concentration in five injections of System suitability. (μg/mL)

A_{STD1}: The average peak area in five injections of System suitability.

A_{SPL.}: The peak area of sample solution

C_{SPL}: The concentration of sample solution (μg/mL)

Pr: the reference standard assay

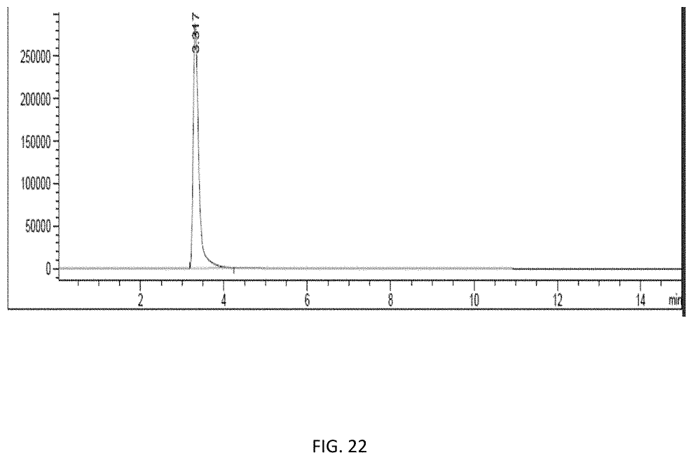

FIG. 22 shows a typical chromatogram result for the compound of Formula (X) generated from batch 201902.

The compound of Formula (X) was collected at a run time of ˜3.3 minutes with an RRT of 1.00. The result showed a 94.0% match between the compound of Formula (X) and the standard.

### LC-MS Serine Assay

Chemical purity was further analyzed by LC-MS using serine as a reference standard and using distilled water, acetonitrile, and formic acid as solvents. A 250*4.6 mm 5 μm column sold under the trade name ECLIPSE XDB C18 by Agilent Technologies, Inc., headquartered in Santa Clara, Calif. was used. Acetonitrile (solvent A) and 0.1% formic acid in water (solvent B) was run according to the gradients shown in Table 22 at a flow rate of 0.6 mL/min, with a column temperature of 25° C., an inject volume of 5 μL, and a run time of 15 minutes. A SIM model with a SIM Ion of 103, positive polarity, and a fragmentor value of 70 was set.

TABLE 22
The gradient table for the LC-MS serine run.

| Time (min) | A % | B % |
|---|---|---|
| 0 | 95 | 5 |
| 8 | 5 | 95 |
| 11 | 5 | 95 |
| 11.1 | 95 | 5 |
| 15 | 95 | 5 |

With water as the diluent, a standard solution and sample solution were made. The standard solution comprises 20 mg of serine in a 100 ml volumetric flask and dilute to volume with water. 1 ml of the stock was then transferred into a 100 ml volumetric flask and diluted to volume with water. The sample solution comprises 20 mg of the compound of Formula (X) from batch 201902 in a 100 ml volumetric flask dilute to volume with water. The sequence of solution runs is shown in Table 23.

TABLE 23
Table of sequence runs for LC-MS serine analysis.

| | | Inject ion | |
|---|---|---|---|
| No. | Solution | number | System suitability criteria |
| 1 | Diluent | ≤1 | No interference |
| 2 | Working standard | 5 | RSD ≤ 5.0% |
| | solution 1 | | |
| 3 | Working standard | 1 | Recovery rate 95%-105% |
| | solution 2 | | |
| 4 | Test solution 1 | 1 | / |
| 5 | Test solution 2 | 1 | / |
| 6 | Working standard | 1 | Recovery rate 95%-105% |
| | solution 1 | | |

The following calculation was made based on the LC-MS run:

Assay (%)=(C_{STD1}×A_{SPL}×Pᵣ)/(C_{SPL}×A_{STD1})×100%

### Where

C_{STD1}: The concentration in five injections of System suitability. (μg/mL)

A_{STD1}: The average peak area in five injections of System suitability.

A_{SPL.}: The peak area of sample solution

C_{SPL}: The concentration of sample solution (μg/mL)

Pr: the reference standard assay

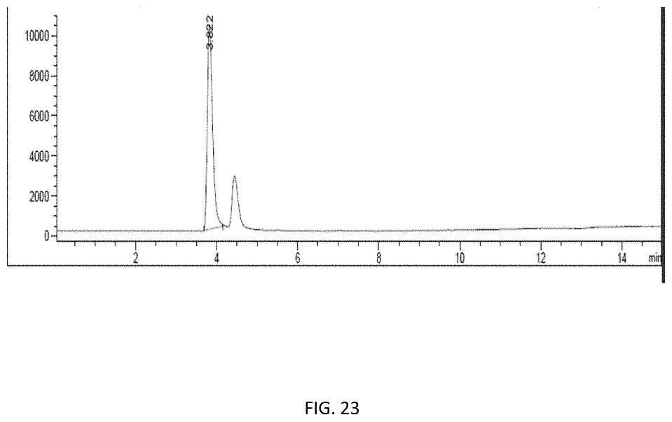

FIG. 23 shows a typical chromatogram result for the serine run.

The compound of Formula (X) was collected at a run time of ˜4.4 minutes with an RRT of 1.17. Serine was collected at a run time of ˜3.8 minutes with an RRT of 1.00. The results showed a 0.80% match between the compound of Formula (X) and serine.

### LC-MS Succinic Acid Assay

Chemical purity was further analyzed by LC-MS using succinic acid as a reference standard and using distilled water, acetonitrile, and formic acid as solvents. A 250*4.6 mm 5 μm column sold under the trade name ECLIPSE XDB C18 by Agilent Technologies, Inc., headquartered in Santa Clara, Calif. was used. Acetonitrile (solvent A) and 0.1% formic acid in water (solvent B) was run according to the gradients shown in Table 24 at a flow rate of 0.5 mL/min, with a column temperature of 25° C., an inject volume of 5 μL, and a run time of 15 minutes. A SIM model with a SIM Ion of 117, negative polarity, and a fragmentor value of 70 was set.

TABLE 24
The gradient table for the LC-MS serine run.

| Time (min) | A % | B % |
|---|---|---|
| 0 | 95 | 5 |
| 8 | 5 | 95 |
| 11 | 5 | 95 |
| 11.1 | 95 | 5 |
| 15 | 95 | 5 |

With water as the diluent, a standard solution and sample solution were made. The standard solution comprises 20 mg of succinic acid in a 100 ml volumetric flask and dilute to volume with water. 1 ml of the stock was then transferred into a 100 ml volumetric flask and diluted to volume with water. The sample solution comprises 20 mg of the compound of Formula (X) from batch 201902 in a 100 ml volumetric flask dilute to volume with water. The sequence of solution runs is shown in Table 25.

TABLE 25
Table of sequence runs for LC-MS serine analysis.

| | | Inject ion | |
|---|---|---|---|
| No. | Solution | number | System suitability criteria |
| 1 | Diluent | ≤1 | No interference |
| 2 | Working standard | 5 | RSD ≤ 5.0% |
| | solution 1 | | |
| 3 | Working standard | 1 | Recovery rate 95%-105% |
| | solution 2 | | |
| 4 | Test solution 1 | 1 | / |
| 5 | Test solution 2 | 1 | / |
| 6 | Working standard | 1 | Recovery rate 95%-105% |
| | solution 1 | | |

The following calculation was made based on the LC-MS run:

Assay (%)=(C_{STD1}×A_{SPL}×Pᵣ)/(C_{SPL}×A_{STD1})×100%

### Where

C_{STD1}: The concentration in five injections of System suitability. (μg/mL)

A_{STD1}: The average peak area in five injections of System suitability.

A_{SPL.}: The peak area of sample solution

C_{SPL}: The concentration of sample solution (μg/mL)

Pr: the reference standard assay

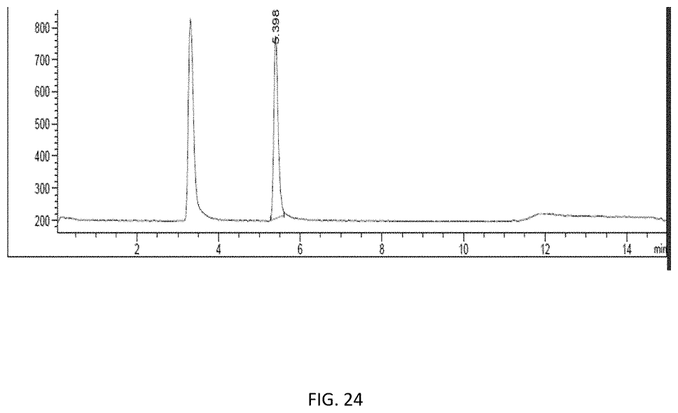

FIG. 24 shows a typical chromatogram result for the succinic acid run.

The compound of Formula (X) was collected at a run time of ˜3.4 minutes with an RRT of 0.61. Succinic acid was collected at a run time of ˜5.4 minutes with an RRT of 1.00. The results showed a 0.64% match between the compound of Formula (X) and succinic acid.

### Chirality Assay

Chirality was further analyzed by HPLC using distilled water and perchloric acid as solvents. A 150*4.0 mm 5 μm CR (+) column was used. A mobile phase of pH of 1.0 aqueous HClO, with a flow rate of 0.4 mL/minute, a 205 nm UV detector, a column temperature of 35° C., an inject volume of 10 μL, and a run time of 20 minutes was used.

With water as the diluent, a positioning solution and two testing solutions were made. The standard solution comprises 50 mg of a compound of Formula (X)-R and 50 mg of a compound of Formula (X)-S mixed in a 50 ml volumetric flask and diluted to volume with water. The first testing solution comprises 100 mg of the sample compound of Formula (X) from batch 201901 in a 50 ml volumetric flask and diluted to volume with water. The second testing solution comprises 100 mg of the sample compound of Formula (X) from batch 201902 in a 50 ml volumetric flask and diluted to volume with water.

The following calculation was made based on the chirality analysis:

$$ee \text{:} \frac{{A}_{RB 6011 - S - {A}_{RB 6011 - R}}}{{A}_{RB 6011 - S + {A}_{RB 6011 - R}}} * 100 \%$$

### Where

A_{RB6011-S}—Area of compound of Formula (X)-S

A_{RB6011-R}—Area of compound of Formula (X)-R

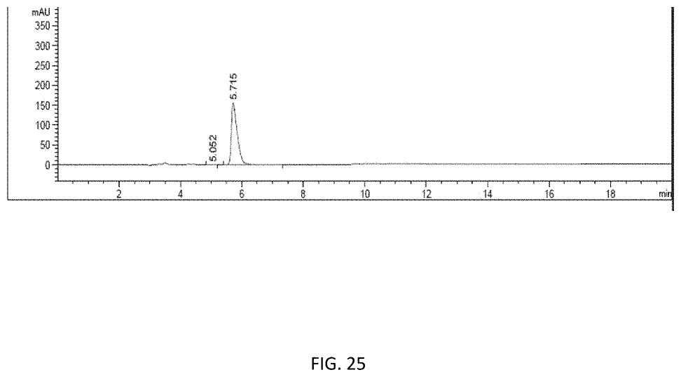

FIG. 25 shows a typical chromatogram result for batch 201901.

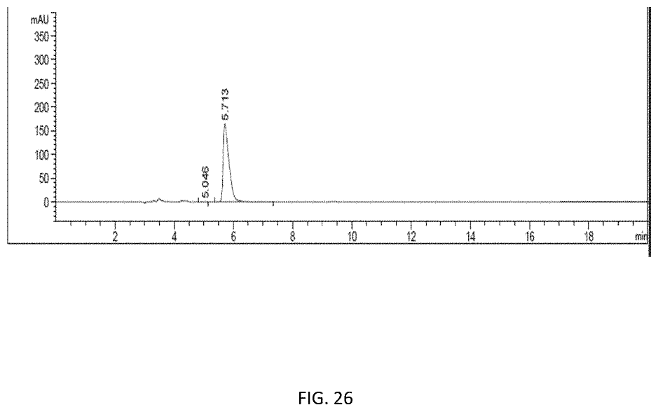

FIG. 26 shows a typical chromatogram result for batch 201902.

The compound of Formula (X)-R was collected at a run time of ˜5.0 minutes with an RRT of 0.88. The compound of Formula (X)-S was collected at a run time of ˜5.7 minutes with an RRT of 1.00. Calculations for batch 201901 resulted in an ee of 99.914%. Calculations for batch 201902 resulted in an ee of 99.934%. Averaging the results produced an ee of ˜99.9%.

### Karl Fischer Moisture Assay

The compound of Formula (X) was further analyzed by a Karl Fischer assay using methanol as a reagent and a Karl Fischer moisture titrator.

About 40 mL of methanol was pumped into the measuring cup for blank titration. A test sample of about 0.1 g of the compound of Formula (X) was placed into a measuring cup, and the moisture titrated with the test sample, the weight of the added test sample recorded, and the concentration and consumption of Karl Fischer titrate at the time of measurement, the volume of Karl Fischer's fluid, and measured moisture data recorded.

The following calculation was made based on the moisture assay:

$$KF = \frac{V \times F}{W} \times 100 \%$$

### Where

V—The volume of Karl Fischer titrate consumed in the test, mL

F—Equivalent to the weight of water per mg of Karl Fischer titrate, mg/mL

W—weight of the test sample, mg

Results indicated 0.15% moisture on a first run, 0.11% residual ignition, and 0.001 residue heavy metal (Pd).

### Residual Solvents Assay

A residual solvents assay was conducted on the compound of Formula (X) using solvents of AR grade acetone, ethyl acetate, methyl tertiary butyl ether, tetradrofuron, and n-Heptane, and HPLC grade n,n-Dimethylformamide as a diluent.

A 30 m*0.32 mm 1.8 μm column sold under the trade name DB-624 by Agilent Technologies, Inc., headquartered in Santa Clara, Calif. was used. Parameters were set with a flow rate of 1.5 mL/min, a split ratio of 10:1, an inlet temperature of 240° C., a detector temperature of 260° C., a gas carrier N₂, a headspace sampler, an over temperature of 100° C., and injector temperature of 110° C., an transfer temperature of ° C., a shake time of 45 minutes, and a cycle time of 55 minutes. The series of temperatures are summarized in Table 26.

TABLE 26
Temperature cycle times

| | Rate (° C. /min) | Temperature (° C.) | Hold time (min) |
|---|---|---|---|
| Oven | N/A | 40.0 | 2.0 |
| temperature | 8.0 | 120.0 | 2.0 |
| | 15.0 | 250.0 | 5.0 |

A standard stock solution was prepared with 5000.0 mg of acetone, 5000.0 mg of methyl tertiary butyl ether, 720.0 mg of tetrahydrofuran, and 5000 mg of n-Heptane in a 100 ml flask diluted with DMF, stirred and mixed. Two working solutions were prepared with 1.0 ml of standard stock solution in a 100 ml flask diluted with dilute solution, stirred and mixed. Two test solutions were prepared with 200 mg of the compound of Formula (X) from batch 201901 and 201902, respectively, in a vial with 20 ml of headspace and 2 ml of DMF. The sequence of solution runs is shown in Table 27.

TABLE 27
Table of sequence runs for LC-MS serine analysis.

| No. | Solution | Number of injection |
|---|---|---|
| 1 | Diluent | ≥1 |
| 2 | Working standard | 5 |
| | solution 1 | |
| 3 | Working standard | 1 |
| | solution 2 | |
| 4 | Test solution 1 | 1 |
| 5 | Test solution 2 | 1 |

The following calculation was made based on the moisture assay:

Residual solvent (ppm)=(Wr×As×Ds×Pr×10)/(Ws×Ar×Dr×10000)×1000000

### Where

Wr: the weight of standards (g);

Ws: The weight of samples (g);

Ar: The area of reference;

As: The area of sample;

Dr: The reference dilute volume;

Ds: The sample dilute volume;

Pr: The weight of reference.

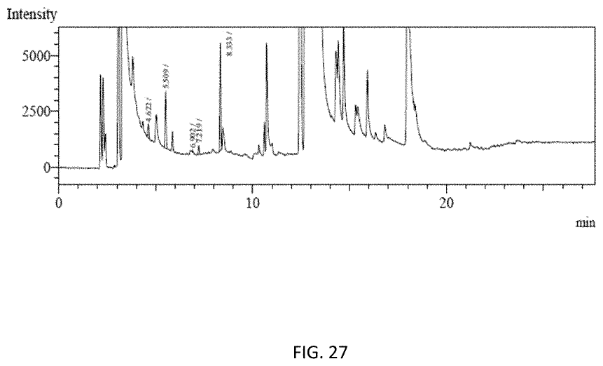

FIG. 27 shows a typical chromatogram of the residual assay runs.

Acetone was collected at a run time of ˜4.7 minutes at 6 ppm. Ethyl acetate was collected at a run time of ˜5.3 minutes at 1 ppm. Methyl tertiary butyl ether was collected at a run time of ˜6.7 minutes at 8 ppm. Tetrahydrofuran was collected at a run time of ˜7.1 minutes at 4 ppm. n-Heptane was collected at a run time of ˜8.2 minutes at 8 ppm. N,N-dimethyl formamide was collected at ˜15.3 minutes.

Quality details for the compound of Formula (X) are summarized in Table 28.

TABLE 28
Quality comparison of batches of a compound of Formula (X)

| Test Item | | Specification | T0419-52-03 Result | T0419-20190 Result |
|---|---|---|---|---|
| Appearance | | Visual inspection | Off-white solid | Off-white solid |
| Identification (¹H-NMR) | | Conforms to the | Conforms | Conforms |
| | | reference standard | | |
| Identification (LC-MS) | | Conforms to the | Conforms | Conforms |
| | | reference standard | | |
| Specific Optical Rotation | | Report | +25.834° | +25.570° |
| Purity (HPLC, % a/a) | | NLT 95.0% | 97.0% | 97.3% |
| Individual | Impurity | Report | 0.15% | 0.17% |
| unknown | RRT 0.56 | | | |
| impurity | Impurity | Report | 0.36% | 0.20% |
| | RRT0.65 | | | |
| | Impurity | Report | N.D. | 0.12% |
| | RTT1.53 | | | |
| | Impurity | Report | 0.43% | 0.65% |
| | RTT1.79 | | | |
| | Impurity | Report | N.D. | 0.13% |
| | RTT2.21 | | | |
| | Impurity | Report | 0.23% | N.D. |
| | RTT2.29 | | | |
| | Impurity | Report | N.D. | 0.15% |
| | RTT2.33 | | | |
| | Impurity | Report | N.D. | 0.44% |
| | RTT2.71 | | | |
| | Impurity | Report | 0.29% | N.D. |
| | RTT4.86 | | | |
| | Impurity | Report | 0.24% | 0.37% |
| | RTT5.04 | | | |
| | Impurity | Report | N.D. | 0.16% |
| | RTT5.21 | | | |
| | Impurity | Report | 0.25% | 0.23% |
| | RTT5.56 | | | |
| | Impurity | Report | 0.18% | N.D. |
| | RTT6.47 | | | |
| | Impurity | Report | 0.07% | N.D. |
| | RTT6.50 | | | |
| | Impurity | Report | 0.48% | 0.12% |
| | RTT6.70 | | | |
| | Impurity | Report | 0.29% | N.D. |
| | RTT6.92 | | | |
| Individual | Pyridine | Report | N.D. | N.D. |
| known | RTT 0.74 | | | |
| impurity | DMAP RRT | Report | N.D. | N.D. |
| | 3.69 | | | |
| | DBU RRT | Report | N.D. | N.D. |
| | 4.38 | | | |
| | RB6011-SM1 | Report | N.D. | N.D. |
| | RRT 6.69 | | | |
| | RB6011-IM295 | Report | 0.05% | N.D. |
| | RRT 6.98 | | | |
| | RB6011-INT1 | Report | N.D. | N.D. |
| | RRT 7.86 | | | |
| | RB6011-INT2 | Report | N.D. | N.D. |
| | RTT 7.92 | | | |
| | Toluene | Report | N.D. | N.D. |
| | RRT 7.98 | | | |
| Assay (LCMS, % w/w) | | Report | 94.6% | 94.0% |
| Impurities | L-Serine | Report | 0.76% | 0.80% |
| (LC-MS % w/w) | Succinic acid | Report | 0.32% | 0.64% |
| Chiral Purity (HPLC, ee) | | Report | 99.6% | 99.9% |
| Water (K.F., % w/w) | | Report | 0.93% | 0.15% |
| Residue on Ignition | | Report | 0.08% | 0.11% |
| Heavy metal (Pd) | | Report | 0.02% | 0.001% |
| Residual solvents | Acetone | NMT 5000 ppm | 11 ppm | 6 ppm |
| (GC-HS % w/w) | Ethyl acetate | NMT 5000 ppm | 6 ppm | 1 ppm |
| | Methyl Tertiary | NMT 5000 ppm | 37 ppm | 8 ppm |
| | Butyl Ether | | | |
| | Tetrahydrofuran | NMT 720 ppm | 4 ppm | 4 ppm |
| | n-Heptane | NMT 5000 ppm | 27 ppm | 8 ppm |

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

What is claimed is:

1. A process for preparing a compound of Formula (X): 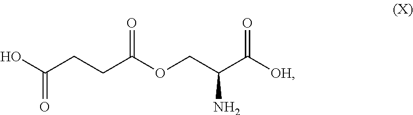 [SMILES: N[C@@H](COC(=O)CCC(=O)O)C(=O)O]
comprising the steps of:
(i) reacting a compound of Formula (I): 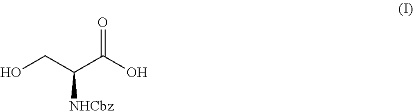 [SMILES: O=C(N[C@@H](CO)C(=O)O)OCc1ccccc1]
in a mixture comprising the compound of Formula (I), 1,8-Diazabicyclo[5.4.0]undec-7-ene (“DBU”) and Benzyl bromide (BnBr) to produce a compound of Formula (II): 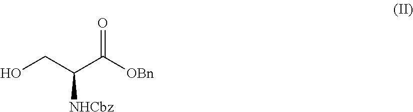 [SMILES: O=C(N[C@@H](CO)C(=O)OCc1ccccc1)OCc1ccccc1]
(ii) reacting the compound of Formula (II) with a compound of Formula (III): 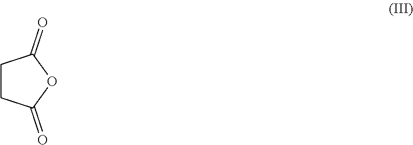
to produce the compound of Formula (IV): 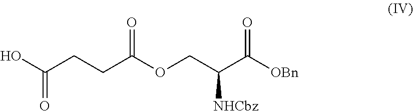 [SMILES: O=C(O)CCC(=O)OC[C@H](NC(=O)OCc1ccccc1)C(=O)OCc1ccccc1]
(iii) reacting the compound of Formula (IV) with Pd(OH)₂/C to form the compound of Formula (X):

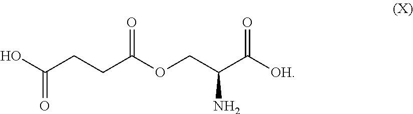 [SMILES: N[C@@H](COC(=O)CCC(=O)O)C(=O)O]

2. The process of claim 1, wherein reacting the compound of Formula (II) with a compound of formula (III) is carried out in the presence of pyridine.

3. The process of claim 2, wherein reacting the compound of Formula (II) with a compound of formula (III) is carried out in the presence of 4-Dimethylaminopyrodine (DMAP).

4. The process of claim 3, wherein reacting the compound of Formula (II) with a compound of formula (III) is carried out in the presence of tetrahydrofuran (THF).

5. The process of claim 1, reacting the compound of Formula (IV) with Pd(OH)₂/C is carried out in the presence of THF.

6. The process of claim 1, wherein reacting the compound of Formula (IV) with Pd(OH)₂/C to form the compound of Formula (X) first forms an intermediate compound of Formula (V):

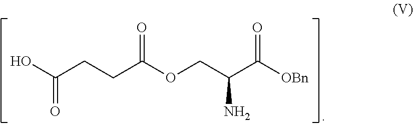

7. The process of claim 1, wherein reacting the compound of Formula (IV) with Pd(OH)₂/C produces methyl benzene and CO₂.

8. The process of claim 1, wherein the compound of Formula (X) prepared is a Type A crystalline polymorph of Formula (X).

9. A pharmaceutical composition comprising a compound of Formula (X): 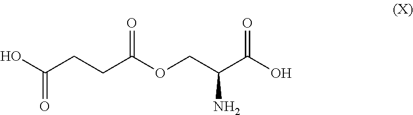 [SMILES: N[C@@H](COC(=O)CCC(=O)O)C(=O)O]
and a pharmaceutically acceptable carrier.

10. The composition of claim 9, wherein the composition is formulated for oral administration.

11. The composition of claim 9, wherein the composition is formulated as a single unit dosage.

12. The composition of claim 9, wherein the composition is formulated as divided dosages.

13. A method of treating a condition in a subject, the method comprising providing to a subject having, or at risk of developing, a condition a composition comprising a therapeutically effective amount of a compound of Formula (X):

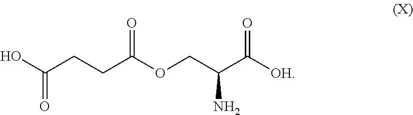 [SMILES: N[C@@H](COC(=O)CCC(=O)O)C(=O)O]

14. The method of claim 13, wherein the composition is provided orally.

15. The method of claim 13, wherein the condition is a condition associated with abnormal TCA cycle metabolism.

16. The method of claim 15, wherein the condition is an inherited disorder, 2-oxoglutaric aciduria, fumarase deficiency, succinyl-CoA synthetase efficiency, a neurodegenerative disorder, Amyotrophic Lateral Sclerosis, Alzheimer's disease, Parkinson's disease, Huntington's disease, a cancer, pancreatic cancer, kidney cancer, cervical cancer, prostate cancer, muscle cancer, gastric cancer, colon cancer, glioblastoma, glioma, paraganglioma, leukemia, liver cancer, breast cancer, carcinoma, neuroblastoma, an energetic disorder, refractory epilepsy, propionic academia (PA), methylmalonic academia (MMA), a long chain fatty acid oxidation disorder, succinyl CoA lyase deficiency, pyruvate carboxylase deficiency, mitochondrial respiratory chain deficiency, glutaric academia type 1 or type 2 a neurological disease, disorder or condition, a pain or fatigue disease, muscular dystrophy (e.g. Duchenne's muscular dystrophy, and Becker's muscular dystrophy), mitochondrial myopathy, mitochondrial encephalomyopathy lactic acidosis and stroke-like syndrome (MELAS), myoclonic epilepsy and ragged-red fibers (MERRF), a mitochondrial associated disease, or a disorder related to POLG mutation.
